(19) United States
(12) Patent Application Publication
(10) Pub. No.: US 2025/0161236 A1
(43) Pub. Date: May 22, 2025

(54) THYMOQUINONE AND BLACK SEED COMPOSITIONS FOR TREATING NEUROENDOCRINE CANCER IN COMBINATION WITH IMMUNOTHERAPY

(71) Applicant: Novatek Pharmaceuticals, Inc., Pearland, TX (US)

(72) Inventors: Abdulhafez Selim, Pearland, TX (US); Ahmed Omar Kaseb, Pearland, TX (US); Mohamed Kaseb, Pearland, TX (US); Amr Mohamed, Pearland, TX (US)

(73) Assignee: Novatek Pharmaceuticals, Inc., Pearland, TX (US)

(21) Appl. No.: 18/954,218

(22) Filed: Nov. 20, 2024

Related U.S. Application Data
(60) Provisional application No. 63/601,182, filed on Nov. 20, 2023.

(51) Int. Cl. A61K 31/122; A61K 36/71; A61K 39/395; A61P 35/00

(52) U.S. Cl. CPC A61K 31/122; A61K 36/71; A61K 39/3955; A61P 35/00

(57) ABSTRACT

Composition and methods for immunotherapy of a cancer patient, comprising administering a black seed oil composition or one or more components thereof, which can include thymoquinone, oleic acid, linoleic acid, palmitic acid, eicosapentaenoic acid, or docosahexaenoic acid. The composition or components can be administered in combination with one or more immune checkpoint inhibitors, including anti-CLA-4 agents and anti-PD-1 agents. The cancers targeted by the compositions and combinations include neuroendocrine cancers, and, in particular, high grade neoplasms and poorly differentiated neuroendocrine carcinomas, including advanced or metastatic neuroendocrine carcinomas and, more particularly, refractory advanced or metastatic gastroenteropancreatic neuroendocrine carcinomas.

### FIELD

The present disclosure generally relates to the use of thymoquinone and black seed oil compositions, as well as other ingredients, in combination with immunotherapy agents to treat neuroendocrine cancers, including neuroendocrine tumors, neuroendocrine carcinomas, and other malignancies.

### BACKGROUND

Neuroendocrine cancers, also referred to as neuroendocrine neoplasms (NENs), originate in neuroendocrine (NE) cells, which share traits of both hormone-producing endocrine cells and nerve cells. NE cells are widely distributed throughout the body, found in the pituitary, parathyroid, thyroid, and adrenal glands, for example, and dispersed in the digestive and respiratory tracts. In response to neuronal input, NE cells release messenger molecules, e.g., hormones, into the blood. NENs can arise from any endocrine region but most frequently arise in the lungs (pulmonary NENs). They also arise in extrapulmonary regions (EP-NENs), most commonly the gastroenteropancreatic (GEP) tract (GEP-NENs). Díez et al. 2013, Ann. Gastroenterol. 26, 29-36.

Accordingly, NENs comprise a heterogeneous family of malignancies, and they show broad and complex clinical behaviors. A recent SEER study indicates a significant rise in NENs over the past decade in conjunction with advances in early-stage detection techniques such as endoscopy and imaging. See, e.g., Dasari et al. 2017, JAMA Oncol. 3, 1335-1342. Classifications based on histologic differentiation and grade have divided NENs into well-differentiated neuroendocrine tumors (NETs) and poorly differentiated neuroendocrine carcinomas (NECs). Low-grade NETs are generally indolent. In contrast, poorly differentiated NECs are aggressive malignancies, with nearly half of patients presenting with synchronous metastatic disease. Extra-pulmonary NECs (EP-NECs or EP-NECAs) are particularly lethal, with patients predominantly having metastatic disease at diagnosis and a median life expectancy of less than a year. Dasari et al. 2018, Cancer 124, 807-815; Garcia-Carbonero et al. 2016, Neuroendocrinology. 103, 186-194.

Approved first-line therapy of NECs is similar to small cell lung cancer, typically comprising a platinum combination chemotherapy, such as etoposide with carboplatin (or cisplatin). Patta et al. 2011, Anticancer Res. 31, 975-978; Zhu et al. 2015, J. Gastrointest. Canc. 46, 166-169. However, in second line settings for NEC patients, e.g., after progression on etoposide plus platinum therapy, there are no current FDA-approved treatments or established standard of care options for NEC patients. Consequently, survival outcomes for such refractory NEC patients can be as short as a few months for aggressive malignancies. Patta and Fakih, 2011, Anticancer Res. 31, 975-978; Zhu et al. 2015, J. Gastrointest. Cancer 46, 166-169.

While research has shed some light on the underlying pathology and mutations associated with NECs, these mechanistic insights have not yet led to proven targetable therapies. Similarly, while immunotherapy based on immune checkpoint inhibitors (ICPIs), such as programmed cell death protein 1 (PD-1), its ligand—programmed cell death protein ligand 1 (PD-L1), and cytotoxic T lymphocyte antigen 4 (CTLA-4) has shown striking effects in treating other cancers, immunotherapy alone has not provided substantial benefits in treating NEC patients, including those refractory to first-line etoposide-platinum therapy.

Thus, there is an urgent need for improved therapeutic options to combat deadly neuroendocrine cancers, including high grade NENs and extrapulmonary NECs, particularly in patients who are refractory to first-line regimens. The present disclosure addresses these and other needs, partly by providing thymoquinone (TQ) compositions comprising black seed oil or other components for use in combination with immunotherapy to treat neuroendocrine cancers, including refractory extrapulmonary NECs.

### SUMMARY

The present disclosure provides compositions and methods for treating cancers, and more particularly, provides methods for treating a neuroendocrine neoplasm (NEN) in a patient by administering a thymoquinone (TQ) composition in combination with an immunotherapy. In embodiments, the methods lead to improvement in treatment efficacy measurements. In embodiments, the methods lead to increases in T cell levels. In embodiments, the methods are associated with specific genetic profiles in the patient.

In embodiments, the NEN is a pulmonary NEN or extrapulmonary NEN, as described further herein. The NEN may include a well-differentiated neuroendocrine tumor (NET) or poorly differentiated neuroendocrine carcinoma (NEC). The NEC may be a pulmonary NEC; an extrapulmonary NEC (EP-NEC), including one arising from the gastroenteropancreatic tract (GEP-NEC); or an NEC of unknown origin. In embodiments, the NEN may be a mixed neuroendocrine-non-neuroendocrine neoplasm (MiNEN), including a mixed neuroendocrine-nonneuroendocrine carcinoma. In embodiments, the NEN (including a NET or NEC) may be stage I, II, III, or IV, and more particularly, is an advanced stage. In embodiments, the pulmonary NEC or EP-NEC is advanced or has metastasized.

In embodiments, patients have previously received one or more lines of therapies, as described further herein, such as a platinum-based combination chemotherapy, and have become refractory, intolerant, or otherwise progressed.

In embodiments, the TQ composition comprises black seed oil (BSO), as described further herein. In embodiments, the BSO has been standardized with respect to amounts of TQ, as well as palmitic acid, oleic acid, and linoleic acid, or other components. The TQ composition may also include defined ranges of other components, which may include antioxidants such as eicosapentaenoic acid or docosahexaenoic acid.

In embodiments, the TQ composition is orally administered as a liquid formulation or a solid dosage formulation comprising BSO or other components, as described herein. More particularly, the solid dosage formulation may be an enteric capsule comprising BSO and include an enteric component incorporated directly into the capsule. In embodiments, the solid dosage formulation is a standardized formulation, such as NP-101 (TQ Formula).

In embodiments, the TQ composition is administered in a total daily amount of at least 3 grams (or less) of BSO, and in a total daily amount of at least 50 mg (or less) of TQ, as described further herein.

In embodiments, immunotherapies include administering one or more immune checkpoint inhibitors, as described in detail herein, and can include an anti-CTLA-4 antibody such as ipilimumab, and an anti-PD-1 antibody such as nivolumab.

In particular embodiments, the present disclosure is directed to a method for treating an NEC, and an EP-NEC in particular, by administering a TQ composition in combination with an immunotherapy comprising a PD-1 inhibitor and a CTLA-4 inhibitor, as described herein. More particularly, the present disclosure is directed to a method for treating an advanced or metastatic EP-NEC, including a GEP-NEC, comprising administering a thymoquinone (TQ) composition in combination with dual immune checkpoint inhibitors (ICPIs) comprising an anti-CTLA-4 antibody such as ipilimumab and an anti-PD-1 antibody such as nivolumab.

### BRIEF DESCRIPTION OF FIGURES

For a more complete understanding of the invention, reference is now made to the Detailed Description and Examples in conjunction with the accompanying figures. This patent application contains at least one drawing executed in color. Copies of this parent application or patent application publication with color drawings will be provided by the Patent Office upon request and payment of the necessary fee.

FIGS. 1A-1D. Anti-tumor activity of thymoquinone (TQ) in human NEN cellular models. (A) Effect of TQ on viability of BON-1 and QCP-1 cells. The values represent 6 replicates per dose from three independent experiments. (B) Effect of TQ on colony formation of NEN cells. Bar graphs show the number of colonies in the presence (+) and absence (−) of the indicated TQ concentrations. (C) Effect of TQ on cellular membrane changes in BON-1 cells associated with apoptosis. (D) Effect of TQ on PARP cleavage in BON-1 cells as a signature of apoptosis.

FIGS. 2A-2B. Synergistic anti-tumor activity of TQ formula (NP-101) plus immune checkpoint inhibitors (ICPIs) in a human NEN cellular model. (A) Viability of BON-1 cells assessed in the presence of TQ alone, the anti-PD1 antibody nivolumab alone, or the combination of TQ and nivolumab. (B) Combination Index (CI) values, indicating a synergistic effect (CI<1) of NP-101 plus nivolumab at most tested doses.

FIG. 3. Anti-tumor activity of orally administered NP-101 in an animal model of neuroendocrine cancer. BON-1 tumor fragments were injected subcutaneously into ICR-SCID mice and tumor volumes were measured in the presence or absence of NP-101 (TQ oil).

FIGS. 4A-4B. Anti-tumor activity of orally administered NP-101 in an animal model of hepatocellular cancer. HepG2 cancer cells were injected into humanized huNOG mice and tumor volumes were measured in the presence or absence of NP-101. (A) Average tumor volume over time. (B) Individual tumor volumes on day 14.

FIGS. 5A-5C. PD-L1 Structure and Drug Binding. (A) PD-L1 dimer (protein model 5N2F) with inhibitor binding site highlighted. (B) High resolution of amino acids interacting with inhibitor in the 5N2F structure. (C) Docking analysis showing interaction of TQ with key residues of the drug binding site of 5N2F.

FIGS. 6A-6B. Anti-tumor activity of TQ plus IPC1s in a 77-year old patient with metastatic neuroendocrine carcinoma of unknown primary origin. Axial T2-weighted MR images (A) before therapy and (B) after therapy demonstrate a significant decrease in multiple liver metastases.

FIGS. 7A-7E. Anti-tumor activity of TQ plus IPC1s in a 75-year old patient with metastatic EP-NEC of hepatobiliary origin. Axial post-contrast fat-suppressed T1 weighted (A) and axial-fused-FDG-PET/CT (B) images at baseline demonstrate a large heterogeneous mass in the right lobe of liver with intense metabolic activity (arrow). Follow-up MRI (C) image after four cycles of therapy reveals a significant decrease in liver tumor burden (arrow). Follow-up MRI (D) and 18-FDG-PET/CT (E) images 2 years after baseline on maintenance therapy indicate complete resolution of mass and metabolic stability.

FIGS. 8A-8D. Anti-tumor activity of TQ plus IPC1s in a 67-year old patient with metastatic colon MiNEN (mixed neuroendocrine-nonneuroendocrine carcinoma) with adenocarcinoma component. Axial-fused-18F-FDG-PET/CT images at baseline demonstrate (A) multiple hypermetabolic retroperitoneal lymph nodes (arrow) and (B) hypermetabolic right psoas mass (arrowhead). Follow-up 18F-FDG-PET/CT images 14 months later demonstrate complete resolution of nodes (C) and right psoas mass (D).

FIGS. 9A-9E. Anti-tumor activity of TQ plus IPC1s in an 85-year old patient with metastatic colon MiNEN with adenocarcinoma component. Axial-contrast enhanced CT images at baseline demonstrate (A) enlarged peripancreatic lymph nodes (arrow) and (B) aortocaval lymph nodes (arrowhead). Follow-up CT images after three cycles of therapy demonstrate a significant decrease in size of (C) peripancreatic lymph nodes (arrow) and (D) aortocaval lymph nodes (arrowhead). CT scan 12 months after therapy (E) shows some disease progression with new portocaval lymphadenopathy (black arrow).

FIGS. 10A-10B. Anti-tumor activity of NP-101 plus IPC1s in a patient with metastatic refractory high-grade neuroendocrine colon carcinoma. (A) CT scan of abdomen at baseline reveals a large aortocaval nodal mass (arrow). (B) CT scan after four cycles of therapy demonstrates significant reduction of aortal nodal mass (arrow). Next Generation Sequencing (NGS): Microsatellite Stable (MSS), Low tumor mutational burden (TMB), Negative PD-L1 Expression.

FIGS. 11A-11B. Anti-tumor activity of NP-101 plus IPC1s in a patient with metastatic refractory high-grade neuroendocrine pancreas carcinoma. (A) CT scan of abdomen at baseline, demonstrating multiple liver metastases (arrows). (B) CT scan after two cycles of therapy, showing decreased size of metastases (arrows). NGS: MSS, Low TMB, Negative PD-L1 Expression.

FIGS. 12A-12B. Anti-tumor activity of NP-101 plus IPC1s in a patient with refractory high-grade neuroendocrine colon carcinoma (A) CT scan of abdomen at baseline, enlarged peripancreatic lymph node (dashed arrow). (B) CT scan after four cycles of therapy, showing decreased size of lymph node (dashed arrow). NGS: High Microsatellite Instability (MSI-H), High TMB, Negative PD-L1 Expression.

FIGS. 13A-13B. Kaplan-Meier survival curves in EP-NEC patients treated with NP-101 plus IPC1s. (A) Progression free survival from onset of treatment (OnTreatment) in all patients (dotted line) and subset with GEP-NEC (solid line). (B) Overall survival from onset of treatment (OnTreatment) in all patients (dotted line) and subset with GEP-NEC (solid line).

FIG. 14. T cell responses in EP-NEC patients before first cycle of treatment (C1), and before fifth cycle of treatment (C5), with NP-101 plus IPCIs. (A) Levels of CD4 positive lymphocytes. (B) Levels of CD8 positive lymphocytes.

### DETAILED DESCRIPTION

The section headings used herein are for organizational purposes only and are not to be construed as limiting the subject matter described.

For the sake of brevity, all publications, including patent applications, patents, and other citations mentioned herein, are incorporated by reference in their entirety to the same extent as if each individual publication, patent or patent application was specifically and individually incorporated by reference. Citation of any such publication, however, shall not be construed as an admission that it is prior art to the present invention.

### Terms and Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms are defined herein for clarity or ready reference so that the present disclosure may be more readily understood, and the inclusion of such definitions herein should not necessarily be construed to represent a substantial difference over what is generally understood in the art.

As used herein, the terms “a,” “an,” and “the” are to be understood as meaning both singular and plural, unless explicitly stated otherwise. Thus, “a,” “an,” and “the” (and grammatical variations thereof where appropriate) refer to one or more. For example, the term “a capsule” includes a plurality of capsules, and mixtures thereof. The presence of broadening words and phrases such as “one or more,” “at least,” “but not limited to” or other like phrases in some instances shall not be read to mean that the narrower case is intended or required in instances where such broadening phrases may be absent. As will become apparent to one of ordinary skill in the art after reading this document, the illustrated embodiments and their various alternatives may be implemented without confinement to the illustrated examples.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof, unless limitation to the singular is explicitly stated.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Moreover, any concentration range, percentage range, ratio range or integer range is to be understood to include the value of any integer within the recited range and, when appropriate, fractions thereof (such as one tenth and one hundredth of an integer), unless otherwise indicated.

As used herein, the term “about” or “approximately” means within an acceptable range for a particular value as determined by one skilled in the art, and may depend in part on how the value is measured or determined, e.g., the limitations of the measurement system or technique. For example, “about” can mean a range of up to 20%, up to 10%, up to 5%, or up to 1% or less on either side of a given value. Thus, where a numerical limitation is used, unless indicated otherwise by the context, “about” means that the numerical value can vary by +20%, and remain within the scope of the disclosed embodiments, and can be further narrowed, if so specified, to +10%, 5%, or +1%. Thus, the term “50 mg TQ” supports the term, if selected, “about 50 mg TQ,” which in turn supports the term, if selected, “50 mg TQ±20%,” “50 mg TQ±10%,” “50 mg TQ±5%,” or “50 mg TQ±1%,” and so on. Alternatively, with respect to biological systems or processes, the term “about” can mean within an order of magnitude, within 5-fold, or within 2-fold on either side of a value. Numerical quantities given herein are approximate unless stated otherwise, meaning that the term “about” or “approximately” can be inferred when not expressly stated. To provide a more concise description, some of the quantitative expressions given herein are not qualified with the term “about.” However, is understood that whether the term “about” is used explicitly or not, every quantity given herein is meant to support both the actual given value and the approximation of such given value that would reasonably be inferred based on the ordinary skill in the art, including equivalents and approximations due to the experimental and/or measurement conditions for such given value. For example, disclosure of “3 gm BSO” or “50 mg TQ” supports “about 3 gm BSO” or “about 50 mg TQ” if so selected. Likewise, disclosure of “at least 1.5 wt %, at least 1.6 wt %, . . . , or at least 3.0 wt % TQ” supports “at least about 1.5 wt %, at least about 1.6 wt %, . . . , or at least about 3.0 wt % TQ” and so on.

The terms “comprising” and “including” are used herein in their open, non-limiting sense. Other terms and phrases used in this document, and variations thereof, unless otherwise expressly stated, should be construed as open ended, as opposed to limiting. Thus, the term “example” is used to provide exemplary instances of the item in discussion, not an exhaustive or limiting list thereof. Similarly, adjectives such as “conventional,” “traditional,” “normal,” or “known” and terms of similar meaning should not be construed as limiting the item described to a given time period or to an item available as of a given time, but they should be read to encompass conventional, traditional, or normal technologies that may be available or known now or at any time in the future. Likewise, where this document refers to technologies that would be apparent or known to one of ordinary skill in the art, such technologies encompass those apparent or known to the skilled artisan now or at any time in the future.

“Administering” refers to the physical introduction of a composition comprising a therapeutic agent to a subject, using any of the various methods and delivery systems known to those skilled in the art. Preferred routes of administration for antibodies (Abs) of the invention include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Alternatively, an Ab of the disclosure can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods. In embodiments, administering refers to oral administration in the case of TQ compositions and parental administration in the case of antibodies used herein.

By way of example, antibodies described in the methods herein are typically administered parenterally, and TQ compositions, including NP-101 (TQ Formula), are typically administered orally as a liquid or more commonly, as a dosage form, such as a tablet, capsule, and more particularly, an enteric soft gel capsule. Moreover, reference to administering a TQ composition in combination with immunotherapy components, such as antibodies, does not mean that the TQ composition must be administered at the same time or with some dosing regimen as the immunotherapy components. Unless explicitly stated otherwise, parameters for administering a composition are independent of the parameters for administering immunotherapy components such as antibodies.

The terms “individual,” “subject,” “participant,” and “patient” are used interchangeably herein and can be a vertebrate, in particular, a mammal, more particularly, a primate (including non-human primates and humans) and include a laboratory animal in the context of a clinical or pre-clinical trial or screening or activity experiment. Thus, as can be readily understood by one of ordinary skill in the art, the formulations of the present invention are particularly suited to administration to any vertebrate, particularly a mammal, and more particularly, a human.

As used in the present disclosure, the term “effective amount” is interchangeable with “therapeutically effective amount” and means an amount or dose of thymoquinone, and/or other active components in the black seed oil, effective in treating the particular disease, condition, or disorder disclosed herein, and thus “treating” includes producing a desired preventative, inhibitory, relieving, or ameliorative effect. In methods of treatment according to the invention, “an effective amount” of any one of the presently described formulations is administered to a subject (e.g., a mammal). The “effective amount” will vary, depending on numerous factors, such as the compound, the disease (and its severity), the treatment desired, and age and weight of the subject, and can be determined by persons of ordinary skill in the art based on the circumstance.

A “therapeutically effective amount” or “therapeutically effective dosage” of a drug or therapeutic agent, such as an Ab or TQ composition of the disclosure, is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug. An anti-cancer agent promotes cancer regression, can also halt cancer progression on imaging, improve quality of life, improve survival outcomes such as time to tumor progression (TTP), progression-free survival (PFS), and overall survival (OS).

By way of further example for the treatment of tumors, a therapeutically effective amount of the drug preferably inhibits cell growth or tumor growth by at least about 20%, more preferably by at least about 40%, even more preferably by at least about 60%, and still more preferably by at least about 80% relative to untreated subjects. In other preferred embodiments of the invention, tumor regression may be observed and continue for a period of at least about 20 days, preferably at least about 40 days, or even more preferably at least about 60 days. Notwithstanding these ultimate measurements of therapeutic effectiveness, evaluation of immunotherapeutic drugs must also make allowance for “immune-related” response patterns.

The term “carrier” refers to an adjuvant, vehicle, or excipients, with which the compound is administered. In certain embodiments, the carrier is a solid carrier. Suitable pharmaceutical carriers include those described in Remington: The Science and Practice of Pharmacy, 23rd Edition (2020).

The term “formulation,” as used herein, is the form in which the dose is to be administered to the subject or patient. The TQ composition, including one containing a black seed oil (BSO) extract, can be administered as part of a formulation that includes non-active agents, such as, for example, enteric components forming part of the shell of a capsule or a soft gel capsule. Such enteric components can also be incorporated directly into the dosage form itself, such as directly into the tablet or capsule itself, or be coated thereon. Thus, a formulation may be administered, for example, in an enteric capsule that includes an enteric component incorporated directly into the capsule, or in an enteric tablet that includes an enteric component incorporated directly into the tablet.

The term “pharmaceutically acceptable,” as used in connection with formulations of the subject disclosure, refers to molecular entities and other ingredients of such formulations that are physiologically tolerable and do not typically produce untoward reactions when administered to an animal (e.g., human) according to their intended mode of administration (i.e., oral).

A “pharmaceutically acceptable excipient” refers to a substance that is non-toxic, biologically tolerable, and otherwise biologically suitable for administration to a subject, such as an inert substance, added to a pharmacological formulation or otherwise used as a vehicle, carrier, or diluents to facilitate administration of an agent and that is compatible therewith. Suitable pharmaceutical carriers include those described in Remington: The Science and Practice of Pharmacy, 23rd Ed. (2020).

As used herein, the term “inert” refers to any inactive ingredient of a described formulation. The definition of “inactive ingredient” as used herein follows that of the U.S. Food and Drug Administration, as defined in 21 C.F.R. 201.3(b)(8), which is any component of a drug product other than the active ingredient.

As used herein, “suitable for oral administration” refers to a sterile, pharmaceutical product, such as a product produced under good manufacturing practices (GMP), as understood in the art, suitable for administration to a subject (e.g., a human subject). As used herein, products suitable for oral administration include liquid dosage forms or formulations. Such products can also be a solid dosage form or a solid dosage formulation, which as used herein, includes formulation (such as capsules and soft gel capsules) that comprise an inner solution, such as black seed oil (BSO).

As used herein, the term “disorder” is used interchangeably with “disease” or “condition.” For example, a pulmonary disorder also means a pulmonary disease or a pulmonary condition, including neoplasms, cancers, carcinomas, tumors, and malignancies. Likewise, an extra-pulmonary disorder also means an extra-pulmonary disease or extra-pulmonary condition, including neoplasms, cancers, carcinomas, tumors, and malignancies.

The terms “treat,” “treating,” and “treatment” cover therapeutic methods directed to a disease-state in a subject and include: (i) preventing the disease-state from occurring, in particular, when the subject is predisposed to the disease-state but has not yet been diagnosed as having it; (ii) inhibiting the disease-state, e.g., arresting its development (progression) or delaying its onset; and (iii) relieving the disease-state, e.g., causing regression of the disease state until a desired endpoint is reached. These terms also include ameliorating a symptom of a disease (e.g., reducing the pain, discomfort, or deficit), wherein such amelioration may be directly affecting the disease (e.g., affecting the disease's cause, transmission, or expression) or not directly affecting the disease.

An “adverse event” (AE) as used herein is any unfavorable and generally unintended or undesirable sign (including an abnormal laboratory finding), symptom, or disease associated with the use of a medical treatment. For example, an adverse event may be associated with activation of the immune system or expansion of immune system cells (e.g., T cells) in response to a treatment. A medical treatment may have one or more associated AEs and each AE may have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (Clq) of the classical complement system.

Antibodies typically bind specifically to their cognate antigen with high affinity, reflected by a dissociation constant (KD) of 10-5 to 10-11 M-1 or less. Any KD greater than about 10-4 M-1 is generally considered to indicate nonspecific binding. As used herein, an Ab that “binds specifically” to an antigen refers to an Ab that binds to the antigen and substantially identical antigens with high affinity, which means having a KD of 10-7 M or less, preferably 10-8 M or less, even more preferably 5x10-9 M or less, and most preferably between 10-8 M and 10-10 M or less, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen. By way of example, an Ab that binds specifically to human PD-1 may also have cross-reactivity with PD-1 antigens from certain primate species but may not cross-react with PD-1 antigens from certain rodent species or with an antigen other than PD-1, e.g., a human PD-L1 antigen.

An “antibody” may be of any immunoglobulin isotype, including IgG (including IgG1, IgG2, IgG3 and IgG4 subtypes), IgA (including IgA1 and IgA2 subtypes), IgM and IgE. The term “antibody” may include, for instance, monoclonal, chimeric, recombinant, deimmunized, affinity matured, humanized and human antibodies, as well as antibodies from other species such as rodents, rabbits, mice, rats, hamsters, goats and llamas. Antibodies may be derived solely from a single source, or may be “chimeric,” that is, different portions of the antibody (such as CDRs, framework regions, variable region, constant region) may be derived from two different antibodies. The definition of “antibody” according to the invention comprises full-length antibodies, also including camelid antibodies, and other immunoglobulins generated by biotechnological or protein engineering methods or processes. An antibody may also be produced in hybridomas. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

The terms “cancer,” “cancerous,” or “malignant” refer to or describe various diseases typically characterized by unregulated cell growth. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer (NK)/T-cell lymphoma, multiple myeloma, acute myelogenous leukemia (AML), and chronic lymphocytic leukemia (CML).

More particularly, the cancer includes a neuroendocrine cancer. With respect to neurocrine growth disorders, the term cancer is interchangeable with “neoplasm” and “malignancy” and can encompass neuroendocrine tumors (NETs), neuroendocrine carcinomas (NECs) and mixed neuroendocrine-non-neuroendocrine neoplasms (MiNENs) of all stages and grades, advanced, and metastatic.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease. In embodiments, immunotherapy refers to cancer immunotherapy, and more particularly, checkpoint inhibitor therapy, such as one based on inhibition of one or more of PD-1, PD-L1, or CTLA-4, as described further herein.

The term “immunoregulatory” refers to a substance, an agent, a signaling pathway or a component thereof that regulates an immune response. “Regulating,” “modifying” or “modulating” an immune response refers to any alteration in a cell of the immune system or in the activity of such cell. Such regulation includes stimulation or suppression of the immune system which may be manifested by an increase or decrease in the number of various cell types, an increase or decrease in the activity of these cells, or any other changes which can occur within the immune system. Both inhibitory and stimulatory immunoregulators have been identified, some of which may have enhanced function in the cancer microenvironment.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response. In embodiments, the TQ compositions disclosed herein can potentiate the effectiveness of immunotherapy in any of the methods herein.

The “Programmed Death-1 (PD-1)” receptor refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. AAH74740.1.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

“CTLA4” or “CTLA-4” (cytotoxic T-lymphocyte-associated protein 4), also known as CD152 (cluster of differentiation 152), is a protein receptor and member of the immunoglobulin superfamily that is expressed by activated T cells and transmits an inhibitory signal to T cells. CTLA-4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands, CTLA-4 transmits an inhibitory signal to T cells, whereas CD28 transmits a stimulatory signal. CTLA-4 is also found in regulatory T cells (Tregs) and contributes to their inhibitory function. T cell activation through the T cell receptor and CD28 can lead to increased expression of CTLA-4. The term “CTLA4” or “CTLA-4” as used herein includes human CTLA4 (hCTLA-4), variants, isoforms, and species homologs of hCTLA-4, and analogs having at least one common epitope with hCTLA-4. The complete hCTLA-4 sequence can be found under GenBank Accession No. P16410.

A “signal transduction pathway” or “signaling pathway” refers to the biochemical relationship between a variety of signal transduction molecules that play a role in the transmission of a signal from one portion of a cell to another portion of the cell. A “cell surface receptor” includes, for example, molecules and complexes of molecules that are located on the surface of a cell and are capable of receiving a signal and transmitting such a signal across the plasma membrane of a cell. An example of a cell surface receptor of the present invention is the PD-1 receptor, which is located on the surface of activated T cells, activated B cells and myeloid cells, and transmits a signal that results in a decrease in tumor-infiltrating lymphocytes and a decrease in T cell proliferation. An “inhibitor” of signaling refers to a compound or agent that antagonizes or reduces the initiation, reception or transmission of a signal, be that signal stimulatory or inhibitory, by any component of a signaling pathway such as a receptor or its ligand.

An “immune-related” response pattern refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional or conventional chemotherapeutic agents would be classified as disease progression and would be synonymous with drug failure. Accordingly, proper evaluation of immunotherapeutic agents may require long-term monitoring of the effects of these agents on the target disease.

A “tumor-infiltrating inflammatory cell” is any type of cell that typically participates in an inflammatory response in a subject and which infiltrates tumor tissue. Such cells include tumor-infiltrating lymphocytes (TILs), macrophages, monocytes, eosinophils, histiocytes and dendritic cells.

Reference will now be made to the embodiments of the present invention, examples of which are illustrated by and described in conjunction with the accompanying examples. While certain embodiments are described herein, it is understood that the described embodiments are not intended to limit the scope of the invention. On the contrary, the present disclosure is intended to cover alternatives, modifications, and equivalents that can be included within the invention as defined by the appended claims.

### Compositions

The present disclosure provides thymoquinone (TQ) compositions for therapeutic use, including uses in treating cancer. Thymoquinone (TQ, C10H1202) is conventionally considered the main bioactive component of black seed or black cumin seed (Nigella sativa, Ranunculaceae family). As disclosed herein, including in the Examples, TQ and BSO show inhibitory effects against neuroendocrine cancer in human cellular models and animal models, and these effects include synergistic action in combination with immunotherapy agents (e.g., immune checkpoint inhibitors). Furthermore, human studies disclosed herein, e.g., Examples 6 and 7, support the use of TQ compositions in combination with immunotherapy (e.g., dual immune checkpoint inhibitors) to treat neuroendocrine cancer, including metastatic and advanced extrapulmonary neuroendocrine carcinomas.

Accordingly, in embodiments, the TQ composition comprises TQ. In embodiments, the TQ composition comprises black seed oil (BSO). In embodiments, the BSO comprises 0.5-3.0 wt % TQ. TQ may be naturally present in the BSO, added exogenously, or both.

In embodiments, the amount of TQ is at least 0.5 wt %, at least 0.75 wt %, at least 1 wt %, at least 1.25 wt %, at least 1.5 wt %, at least 1.6 wt %, at least 1.7 wt %, at least 1.8 wt %, at least 2.0 wt %, at least 2.1 wt %, at least 2.2 wt %, at least 2.3 wt %, at least 2.4 wt %, at least 2.5 wt %, at least 2.6 wt %, at least 2.7 wt %, at least 2.8 wt %, at least 2.9 wt %, or at least 3.0 wt %, based on the total weight of BSO in the formulation.

In embodiments, the amount of TQ comprises about 0.5 wt %, about 0.75 wt %, about 1 wt %, about 1.25 wt %, about 1.5 wt %, about 1.6 wt %, about 1.7 wt %, about 1.8 wt %, about 1.9 wt %, about 2.0 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, or about 3.0 wt % TQ, based on the total weight of BSO in the formulation.

In embodiments, the BSO comprises at least 1.5 wt % TQ. In embodiments, the BSO comprises at least 1.7 wt % TQ. In embodiments, the BSO comprises at least 1.8 wt % TQ. In embodiments, the BSO comprises at least 1.9 wt % TQ. In embodiments, the BSO comprises at least 2.0 wt % TQ. In embodiments, the BSO comprises at least 2.0 wt % TQ. In embodiments, the BSO comprises at least 2.2 wt % TQ. In embodiments, the BSO comprises at least 2.5 wt % TQ. In embodiments, the BSO comprises at least 2.8 wt % TQ. In embodiments, the BSO comprises at least 3.0 wt % TQ.

Dosing amounts of BSO can be adjusted based on the concentration of thymoquinone in the administered BSO formulation.

In the compositions herein, BSO can also contain, in addition to TQ at any of the weight percentages disclosed herein, any number of other components or ingredients, such as palmitic acid (PA), oleic acid (PA), or linoleic acid (PA), as well as one or more antioxidants such as eicosapentaenoic acid (EPA) or docosahexaenoic acid (DHA).

In embodiments, the BSO comprises palmitic acid (PA). In embodiments, the BSO comprises 9-15 wt % PA. PA may be naturally present in the BSO, added exogenously, or both.

In embodiments, the BSO comprises at least 9 wt %, at least 9.5 wt %, at least 10 wt %, at least 10.5 wt %, at least 11 wt %, at least 11.5 wt %, at least 12 wt %, at least 12.5 wt %, at least 13 wt %, at least 13.5 wt %, at least 14 wt %, at least 14.5 wt %, or at least 15.0 wt % PA.

In embodiments, the BSO comprises about 9 wt %, about 9.5 wt %, about 10 wt %, about 10.5 wt %, about 11 wt %, about 11.5 wt %, about 12 wt %, about 12.5 wt %, about 13 wt %, about 13.5 wt %, about 14 wt %, about 14.5 wt %, or about 15.0 wt % PA.

In embodiments, the BSO comprises oleic acid (OA). In embodiments, the BSO comprises 18-30 wt % OA. OA may be naturally present in the BSO, added exogenously, or both.

In embodiments, the BSO comprises at least 18 wt %, at least 18.5 wt %, at least 19 wt %, at least 19.5 wt %, at least 20 wt %, at least 20.5 wt %, at least 21 wt %, at least 21.5 wt %, at least 22 wt %, at least 22.5 wt %, at least 23 wt %, at least 23.5 wt %, at least 24 wt %, at least 24.5 wt %, at least 25 wt %, at least 25.5 wt %, at least 26 wt %, at least 26.5 wt %, at least 27 wt %, at least 27.5 wt %, at least 28 wt %, at least 28.5 wt %, at least 29 wt %, at least 29.5 wt %, or at least 30 wt % OA.

In embodiments, the BSO comprises about 18 wt %, about 18.5 wt %, about 19 wt %, about 19.5 wt %, about 20 wt %, about 20.5 wt %, about 21 wt %, about 21.5 wt %, about 22 wt %, about 22.5 wt %, about 23 wt %, about 23.5 wt %, about 24 wt %, about 24.5 wt %, at least 25 wt %, about 25.5 wt %, about 26 wt %, about 26.5 wt %, about 27 wt %, about 27.5 wt %, about 28 wt %, about 28.5 wt %, about 29 wt %, about 29.5 wt %, or about 30 wt % OA.

In embodiments, the BSO comprises linoleic acid (LA). In embodiments, the BSO comprises 52-68 wt % LA. LA may be naturally present in the BSO, added exogenously, or both.

In embodiments, the BSO comprises at least 52 wt %, at least 52.5 wt %, at least 53 wt %, at least 53.5 wt %, at least 54 wt %, at least 54.5 wt %, at least 55 wt %, at least 55.5 wt %, at least 56 wt %, at least 56.5 wt %, at least 57 wt %, at least 57.5 wt %, at least 58 wt %, at least 58.5 wt %, at least 59 wt %, at least 59.5 wt % LA, at least 60 wt %, at least 60.5 wt %, at least 61 wt %, at least 61.5 wt %, at least 62 wt %, at least 62.5 wt %, at least 63 wt %, at least 63.5 wt %, at least 64 wt %, at least 64.5 wt %, at least 65 wt %, at least 65.5 wt %, at least 66 wt %, at least 66.5 wt %, at least 67 wt %, at least 67.5 wt %, or at least 68 wt % LA.

In embodiments, the BSO comprises about 52 wt %, about 52.5 wt %, about 53 wt %, about 53.5 wt %, about 54 wt %, about 54.5 wt about 55 wt %, about 55.5 wt %, about 56 wt %, about 56.5 wt %, about 57 wt %, about 57.5 wt %, about 58 wt %, about 58.5 wt %, about 59 wt %, about 59.5 wt % LA, about 60 wt %, about 60.5 wt %, about 61 wt %, about 61.5 wt %, about 62 wt %, about 62.5 wt %, about 63 wt %, about 63.5 wt %, about 64 wt %, about 64.5 wt %, about 65 wt %, about 65.5 wt %, about 66 wt %, about 66.5 wt %, about 67 wt %, about 67.5 wt %, or about 68 wt % LA.

In embodiments, the TQ composition comprises an antioxidant. In embodiments, the antioxidant is naturally present in the BSO extract. In embodiments, the antioxidant in the TQ composition is added exogenously to the BSO. In embodiments, the antioxidant in the TQ composition in present in the BSO and added exogenously to the BSO composition. In embodiments, the antioxidant is an omega-3 fatty acid, and more particularly, can include eicosapentaenoic acid (EPA) or docosahexaenoic acid (DHA) or both.

In embodiments, the TQ composition comprises 0.8-6.0 wt % of EPA. In embodiments, the TQ composition comprises at least 0.8 wt %, at least 1.0 wt %, at least 1.5 wt %, at least 2.0 wt %, at least 2.5 wt %, at least 3.0 wt %, at least 3.5 wt %, at least 4.0 wt %, at least 4.5 wt %, at least 5.0 wt %, at least 5.5 wt %, or at least 6.0 wt % EPA. In embodiments, the TQ composition comprises about 0.8 wt %, about 1.0 wt %, about 1.5 wt %, about 2.0 wt %, about 2.5 wt %, about 3.0 wt %, about 3.5 wt %, about 4.0 wt %, about 4.5 wt %, about 5.0 wt %, about 5.5 wt %, or about 6.0 wt % EPA.

In embodiments, the TQ composition comprises 0.4-3.0 wt % docosahexaenoic acid (DHA). In embodiments, the TQ composition comprises at least 0.4 wt %, at least 0.8 wt %, at least 1.2 wt %, at least 1.6 wt %, at least 2 wt %, at least 2.4 wt %, at least 2.8 wt %, or at least 3 wt % DHA. In embodiments, the TQ composition comprises about 0.4 wt %, about 0.8 wt %, about 1.2 wt %, about 1.6 wt %, about 2 wt %, about 2.4 wt %, about 2.8 wt %, or about 3 wt % DHA.

In embodiments, the TQ composition comprises 1.5-2.5 wt % TQ, 9-15 wt % palmitic acid, 18-30 wt % oleic acid, 52-68 wt % linoleic acid, 0.8-6 wt % EPA, and 0.4-3 wt % DHA.

In embodiments, the BSO comprises at least 1.5 wt % TQ, 9-15 wt % palmitic acid, 18-30 wt % oleic acid, and 52-68 wt % linoleic acid. In some aspects, the BSO comprises at least 1.5 wt % TQ, at least 9-15 wt % palmitic acid, at least 18-30 wt % oleic acid, and at least 52-68 wt % linoleic acid. In some aspects, the BSO comprises at least 1.5 wt % TQ, at least 9-15 wt % palmitic acid, at least 18-30 wt % oleic acid, at least 52-68 wt % linoleic acid, at least 0.8-6 wt % EPA, and at least 0.4-3 wt % DHA. In some aspects, the BSO comprises about 1.5 wt % TQ, about 9-15 wt % palmitic acid, about 18-30 wt % oleic acid, about 52-68 wt % linoleic acid, about 0.8-6 wt % EPA, and about 0.4-3 wt % DHA.

In embodiments, the BSO comprises at least 1.7 wt % TQ, 9-15 wt % palmitic acid, 18-30 wt % oleic acid, and 52-68 wt % linoleic acid. In some aspects, the BSO comprises at least 1.7 wt % TQ, at least 9-15 wt % palmitic acid, at least 18-30 wt % oleic acid, and at least 52-68 wt % linoleic acid. In some aspects, the BSO comprises at least 1.7 wt % TQ, at least 9-15 wt % palmitic acid, at least 18-30 wt % oleic acid, at least 52-68 wt % linoleic acid, at least 0.8-6 wt % EPA, and at least 0.4-3 wt % DHA. In some aspects, the BSO comprises about 1.7 wt % TQ, about 9-15 wt % palmitic acid, about 18-30 wt % oleic acid, about 52-68 wt % linoleic acid, about 0.8-6 wt % EPA, and about 0.4-3 wt % DHA.

In embodiments, the BSO comprises at least 1.9 wt % TQ, 9-15 wt % palmitic acid, 18-30 wt % oleic acid, and 52-68 wt % linoleic acid. In some aspects, the BSO comprises at least 1.9 wt % TQ, at least 9-15 wt % palmitic acid, at least 18-30 wt % oleic acid, and at least 52-68 wt % linoleic acid. In some aspects, the BSO comprises at least 1.9 wt % TQ, at least 9-15 wt % palmitic acid, at least 18-30 wt % oleic acid, at least 52-68 wt % linoleic acid, at least 0.8-6 wt % EPA, and at least 0.4-3 wt % DHA. In some aspects, the BSO comprises about 1.9 wt % TQ, about 9-15 wt % palmitic acid, about 18-30 wt % oleic acid, about 52-68 wt % linoleic acid, about 0.8-6 wt % EPA, and about 0.4-3 wt % DHA.

In embodiments, the BSO comprises at least 2 wt % TQ, 9-15 wt % palmitic acid, 18-30 wt % oleic acid, and 52-68 wt % linoleic acid. In some aspects, the BSO comprises at least 2 wt % TQ, at least 9-15 wt % palmitic acid, at least 18-30 wt % oleic acid, and at least 52-68 wt % linoleic acid. In some aspects, the BSO comprises at least 2 wt % TQ, at least 9-15 wt % palmitic acid, at least 18-30 wt % oleic acid, at least 52-68 wt % linoleic acid, at least 0.8-6 wt % EPA, and at least 0.4-3 wt % DHA. In some aspects, the BSO comprises about 2 wt % TQ, about 9-15 wt % palmitic acid, about 18-30 wt % oleic acid, about 52-68 wt % linoleic acid, about 0.8-6 wt % EPA, and about 0.4-3 wt % DHA.

In any of the preceding embodiments, a TQ composition can comprise any one or more of the BSO components described herein. For example, a TQ composition can comprise any one or more of the following: 1.5-2.5 wt % TQ, 9-15 wt % palmitic acid, 18-30 wt % oleic acid, 52-68 wt % linoleic acid, 0.8-6 wt % EPA, and 0.4-3 wt % DHA. For example, a TQ composition can comprise any one or more of the following: at least 1.7 wt % TQ, at least 9-15 wt % palmitic acid, at least 18-30 wt % oleic acid, at least 52-68 wt % linoleic acid, at least 0.8-6 wt % EPA, and at least 0.4-3 wt % DHA. For example, a TQ composition can comprise any one or more of the following: about 1.7 wt % TQ, about 9-15 wt % palmitic acid, about 18-30 wt % oleic acid, about 52-68 wt % linoleic acid, about 0.8-6 wt % EPA, and about 0.4-3 wt % DHA.

### Formulations

In some embodiments, the TQ composition is administered as an inhalation formulation comprising the BSO or BSO components. In embodiments the TQ composition is aerosolized for pulmonary administration. In embodiments, aerosolization of the TQ composition comprises a nebulizer, a pressurized metered-dose inhale, or a drug powder inhaler.

In some embodiments, the TQ composition is orally administered as a liquid formulation comprising the BSO.

In some embodiments, the TQ composition is orally administered in a solid dosage formulation comprising the BSO. As used herein, a “solid dosage formulation” or “solid dosage form,” as intended for oral administration, includes such solid dosage formulations, as disclosed, e.g. in Remington: The Science and Practice of Pharmacy, 23rd Edition (2020) or Dilip M. Parikh, Handbook of Pharmaceutical Granulation Technology, 4^{th} ed (2021) CRC Press, and comprises, but is not limited to, tablets, including chewable tablets, capsules, pills, lozenges, troches, cachets, capsules, and pellets. Accordingly, the term “solid dosage formulation” or “solid dosage form,” as used herein, encompasses formulation (such as capsules and soft gel capsules) that comprise an inner solution, such as black seed oil (BSO).

In some embodiments, the solid dosage formulation is a tablet comprising BSO, a capsule comprising BSO, a hard capsule comprising BSO, or a soft capsule comprising BSO. In particular embodiments, the solid dosage formulation is a soft gel capsule comprising BSO.

In exemplary embodiments, the BSO formulation is formulated in a capsule that, as finally formulated for oral administration, is stable (i.e., does not dissolve) at pH commonly found in the stomach (e.g., pH of about 3 or lower), but readily breaks down (i.e., dissolves or disintegrates) at a higher pH commonly found in the small intestine and further downstream in the digestive tract (e.g., pH of about 5.5 to about 9). Having substantially all the drug release at pH of >5.5 (e.g., 90% solubility of the formulation at pH 6.8), but not at lower pH, may increase the bioavailability of the black seed oil, which may be advantageous to enhancing patient outcome. Additionally, targeting delivery of the drug to the intestine region may find applications in particular indications, such as irritable bowel syndrome. See. e.g., Azad et al. 2020, Pharmaceutics 12, 219, hereby incorporated by reference.

More particularly, the formulations of the instant disclosure remain intact and do not dissolve or disintegrate at low pH as it enters the stomach (e.g., pH of about 3 or lower) and dissolve at an appropriate pH to allow targeting of the duodenum, jejunum or ileum and colon. For example, the formulations, in certain embodiments, can dissolve at pH>5.5 (duodenum targeting), or pH 6-7 (jejunum targeting), or pH above 7 (ileum and colon targeting), while remaining intact and/or not dissolving or disintegrating at lower pHs conditions that are found further upstream in the digestive tract. This can be achieved, for example, based on the capsule, enteric coating and/or banding solution employed. As used herein, and for purposes of brevity, the term “enteric” refers to any one of such formulations.

In certain embodiments, whether a formulation is deemed to be intact, or dissolved or disintegrated, is determined by USP <711>: Dissolution, in either or both of Apparatus 1 (Basket Stirring Element) and/or Apparatus 2 (Paddle Stirring Element).

In embodiments, the instantly disclosed BSO formulations are formulated to provide the instantly disclosed enteric properties via the use of a capsule that is itself enteric,

In other embodiment, the instantly disclosed BSO formulations are formulated to provide the instantly disclosed enteric properties via the use of a capsule that is initially not enteric, but has been modified with an enteric coating prior to loading the black seed oil, so as to provide an enteric formulation as administered by the subject.

Examples of enteric coatings that can find use according to the subject disclosure include enteric components known in the art, including acid-insoluble polymers and film-forming polymers. In exemplary embodiments, the acid-insoluble polymer can also be selected from the group consisting of acrylic and methacrylic acid copolymers, cellulose acetate esters such as phthalate, butyrate, hydroxypropyl methylcellulose phthalate, and salts thereof. In exemplary embodiments, the film-forming polymer is selected from the group consisting of cellulose acetate phthalate, cellulose acetate trimellitate, HPMCP (hypromellose phthalate), HPMCAS (hydroxy propyl methyl cellulose acetate succinate), polyvinyl acetate phthalate (PVAP), and methacrylic acid copolymers.

For example, HPMCP is a phthalic acid ester of hydroxypropyl methylcellulose and has been admitted into the U.S. National Formulary (US/NF). Capsules in which enteric coatings such as HPMCP have been applied are commercially available from, for example, CapsCanada® (Dania Beach, FL), SE Tylose GmbH & Co. KG (Wiesbaden, Germany).

The threshold pH at which, for example, an enteric capsule that includes HPMCP, will dissolve can be controlled, for example, by varying the phthalyl content. In certain exemplary embodiments, the formulation dissolves at, for example, pH>5.5 (duodenum targeting), or pH 6-7 (jejunum targeting), or pH above 7 (ileum and colon targeting).

Other enteric coatings that can find use according to the subject disclosure include coatings that include a polymer having methyl acrylate as a monomer (e.g., a copolymer of methyl acrylate) commercialized with different acidic or alkaline groups to allow for the formulation to remain intact and not dissolve or disintegrate at low pH, but that dissolves at, for example, pH>5.5 (duodenum targeting), or pH 6-7 (jejunum targeting), or pH above 7 (ileum and colon targeting). Methyl acrylate enteric coatings, as described above are commercially available (e.g., Eudagrit® Polymers for Delayed Release such as Eudragit® L 30 D-55, Eudragit® FS 30 D, Eudragit® L and Eudragit® S polymers, commercially available from Evonik Industries AG (Essen, Germany).

The solid dosage form may also include other pharmaceutically acceptable excipients and carriers, as well as other inert components, in addition to the active ingredients.

Accordingly, in some embodiments, the solid dosage formulation is an enteric tablet comprising BSO, an enteric capsule comprising BSO, or an enteric soft gel capsule comprising BSO. The enteric capsule or tablet can further include an enteric component incorporated directly into the tablet or capsule itself. In embodiments, the solid dosage formulation may also include other components or additional amounts of existing components in the BSO or TQ composition.

In some embodiments, the capsule is an enteric capsule that includes an enteric component incorporated directly into the capsule. In some aspects, the enteric capsule remains intact in gastric acid having a pH of about 3 or lower; dissolves or disintegrates at a pH greater than about 5.5; dissolves or disintegrates at a pH of about 6 to about 7, while remaining intact at a pH under about 5.5; or dissolves or disintegrates at a pH of above 7, while remaining intact at a pH under about 6.

In some embodiment, the TQ composition is a solid dosage formulation comprising BSO, wherein the BSO comprises at least 1.7 wt % TQ, 9-15 wt % palmitic acid, 18-30 wt % oleic acid, and 52-68 wt % linoleic acid; the solid dosage formulation is an enteric capsule comprising BSO, and the enteric capsule includes an enteric component incorporated directly into the capsule. In some aspects, the enteric capsule in an enteric soft gel capsule. In some embodiments, the TQ composition is NP-101 (TQ Formula), which ensures standardization of components such as TQ, wherein NP-101 includes, for example, at least 1.7 wt % TQ, 9-15 wt % palmitic acid, 18-30 wt % oleic acid, and 52-68 wt % linoleic acid.

### Dosing

In embodiments, any of the TQ compositions described herein are administered in a total daily amount of at least 1.2 gm BSO, 1.5 gm BSO, at least 1.8 gm BSO, at least 2 gm, at least 2.4 gm BSO, at least 2.5 gm BSO, at least 2.7 BOS, or at least 3 gm BSO.

In embodiments, any of the TQ compositions described herein are administered in a total daily amount of at least 3 gm BSO, or about 3 gm BSO.

In embodiments, a therapeutically effective dosage of the TQ composition is administered in a total daily amount of at least 25 mg, at least 35 mg, at least 40 mg, at least 45 mg, or at least 50 mg TQ. In certain embodiments, the TQ composition is administered in a total daily amount of at least 50 mg TQ. In some embodiments, the TQ composition is NP-101 (TQ Formula).

In embodiments, the TQ Formula is administered in multiple doses. In embodiment, the TQ formula is administered in 5 capsules daily, each capsule comprising 600 mg BSO.

### Other Agents

In embodiments, any of the TQ compositions and formulas disclosed herein are administered to in combination with one or more agents used in immunotherapy, and more particularly, immune checkpoint inhibitors.

In embodiments, any of the treatment methods herein comprise administering a TQ composition in combination with immunotherapy. In embodiments, immunotherapy refers to cancer immunotherapy, and more particularly, checkpoint inhibitor therapy, as described further herein.

Accordingly, in embodiments, the immunotherapy comprises administering a therapeutically effective amount of at least one immune checkpoint inhibitor. Immune checkpoint targets include PD-1, PD-L1, and CTLA-4, and others, such as lymphocyte activation gene-3 (LAG-3), T cell immunoglobulin and mucin-domain containing-3 (TIM-3), T cell immunoglobulin and ITIM domain (TIGIT), V-domain Ig suppressor of T cell activation (VISTA). See, e.g., Shiravand et al. 2022, Curr. Oncol. 29, 3044-3060; Qin et al 2019, Mol. Cancer 18, 155. More particularly, the immune checkpoint inhibitor is an approved drug, such as a PD-1 inhibitor, a PD-L1 inhibitor, or a CTLA-4 inhibitor.

In embodiments, the immunotherapy comprises administering a therapeutically effective amount of a PD-1 inhibitor and a CLTA-4 inhibitor. In some aspects, the PD-1 inhibitor is an anti-PD-1 antibody and the CTLA-4 inhibitor is an anti-CLTA-4 antibody. The anti-PD-1 antibody can comprise nivolumab (Opdivo®), pembrolizumab (Keytruda®), cemiplimab (Libtayo®), dostarlimab (Jemperli®), or retifanlimab (Zynx®), as well as other suitable antibodies. The anti-CTLA-4 antibody can comprise ipilimumab (Yervoy®) or tremelimumab (Imjudo®).

In embodiments, the immunotherapy comprises administering a PD-L1 inhibitor, and more particularly, an anti-PD-L1 antibody. In embodiments, the anti-PD-L1 antibody is atezolizumab (Tecentriq®), avelumab (Bavencio®), or durvalumab (Imfinzi®).

In embodiments, the immunotherapy comprises administering a PD-L1 inhibitor, and more particularly, an anti-PD-L1 antibody.

In embodiments, the immunotherapy comprises administering an anti-PD-1 antibody and an anti-CTLA-4 antibody. In embodiments, the anti-PD-1 antibody is nivolumab and the anti-CTLA-4 antibody is ipilimumab. In embodiments, nivolumab is administered on day 1 of a 3 week cycle for one or more cycles, and more particularly, is administered on day 1 of a 3 week cycle for four cycles and then administered on day 1 of a 2 week cycle for six cycles. In one aspect, nivolumab is administered at a dose of about 3 mg/kg. In embodiments, ipilimumab is administered on day 1 of a 3 week cycle for one or more cycles, and more particularly, for four cycles. In one aspect, ipilimumab is administered at a dose of about 1 mg/kg.

### Methods of Treatment

As described herein, TQ based compositions show anti-cancer activity in neuroendocrine cancer models in vivo and in vitro, including synergistic activity in combination with immunotherapy, e.g., in combination with one or more immune checkpoint inhibitors.

Bioinformatic studies, as discussed in more detail in Example 6, further support a direct role in immunoregulation for BSO components, including TQ and certain fatty acids: docking models indicate significant binding of the TQ and fatty acids to the immune checkpoint target, PD-L1, and, more particular, at a region previously shown to be important for PD-L1 activity. The studies also support a potential role for TQ and certain fatty acids, including omega-3 fatty acids in disrupting the CTLA-4 pathway through inhibition of the associated signaling protein SHP2 (Src homology-2 domain-containing protein tyrosine phosphatase-2)

As further disclosed herein, the use of TQ compositions, in combination with dual ICPIs, shows promising effects in patients with neuroendocrine cancers. In particular, such effects are seen in patients with neuroendocrine carcinomas (NECs), including extra-pulmonary NECs (EP-NECs) with advanced and metastatic disease.

Accordingly, in embodiments, the presently disclosed TQ compositions, including formulation thereof, can be administered to treat any indication or condition in a subject, or administered to healthy subjects seeking to maintain their good health.

More particularly, they can be combined with immunotherapy for the treatment of cancer, including neuroendocrine cancers.

Accordingly, in embodiments, the instantly disclosed formulation are administered in combinations with any of the immunotherapies disclosed herein to treat cancer.

In exemplary embodiments, the presently disclosed formulations are administered to a subject (e.g., orally to a human subject) to treat cancer.

In particular embodiments, the cancer is a neuroendocrine neoplasm (NEN). NENs are a heterogenous family of malignancies that most frequently originate in the pulmonary system (pulmonary NENs). But they also arise in extrapulmonary locations (EP-NENs), most commonly the gastroenteropancreatic tract (GEP-NENs). NENs derive from neuroendocrine (NE) cells, which are distributed widely throughout the body, found in the pituitary, parathyroid, thyroid, and adrenal glands, and dispersed in the digestive or respiratory tracts. In response to neuronal input, neuroendocrine cells release messenger molecules (hormones) into the blood. They produce, store, and in response to neuronal input-secrete bioactive amines and peptide hormones, such as serotonin, insulin, gastrin, vasoactive intestinal peptide, glucagon, chromogranin A, and somatostatin, into the blood. NENs display a distinctive and unique morphology, typically expressing markers of neuroendocrine differentiation, such as chromogranin A in dense core hormone granules, and synaptophysin in the membranes of small presynaptic-like vesicles. The term “neuroendocrine” reflects the sharing of these markers between peptide secreting cells and neuronal cells. See, e.g., Rindi and Wiedenmann, 2012, Nat. Rev. Endocrin. 8, 54-64; Rindi and Inzani, 2020, Endocr. Rel. Cancer. 27, R211-R218.

NENs can be divided into well-differentiated neuroendocrine tumors (NETs) and poorly differentiated neuroendocrine carcinomas (NECs), independent of the site where they arose. See, e.g., Rindi et al. 2018, Mod. Pathol. 31, 1770-1786; Nagtegaal et al. 2020, Histopathology 76, 182-188.

The following chart summarized these characteristics for extrapulmonary NENs arising in the gastroenteropancreatic (GEP) tract (see, e.g., Nagtegaal et al. 2020):

| Differentiation | Grade | Proliferation Indices |
|---|---|---|
| NETs | Low (G1) | Mitotic rate: <2 |
| Well differentiated | | Ki67 index <3% |
| | Intermediate (G2) | Mitotic rate: 2 to 20 |
| | | Ki67 index 3%-20% |
| | High (G3) | Mitotic rate: >20 |
| | | Ki67 index >20% |
| NECs | High (G3) | Mitotic rate: >20 |
| Poorly differentiated | | Ki67 index >20% |
| (large-cell or small-cell type) | | |
| MiNEN | Variable | Variable |
| Variable: Can be poorly | | |
| differentiated or well | | |
| differentiated | | |

In accordance with this classification, well-differentiated NETs are further graded as low (G1), intermediate (G2), or high (G3), on based on two proliferation indices, Miotic rate and Ki-67. As described in Nagtegaal et al, 2020, for example, the Mitotic Rates can be calculated as the number of mitoses/2 mm² as determined by counting in 50 fields of 0.2 mm² (i.e. in a total area of 10 mm²); the Ki-67 proliferation index value is determined by counting at least 500 cells in the regions of highest labelling (hot-spots), which are identified at scanning magnification; and the final grade is based on whichever of the two proliferation indexes places the neoplasm in the higher-grade category.

Poorly differentiated EP-NECs in this chart are not formally graded, but are considered high-grade by definition, e.g., G3, but continue to be separated into small-cell neuroendocrine carcinoma (SC-NEC) and large-cell neuroendocrine carcinoma (LC-NEC) types.

The overall prognosis of NENs, including NETs and NECs, is further dictated by staging: localized (stages I and II), regional spread (stage III), and advanced with distant metastasis (stage IV). Given their poorly differentiated status, NECs are an aggressive subgroup, with rising incidence and mortality. Most patients with EP-NEC, regardless of their primary site, are initially diagnosed with metastatic disease and have poor survival. See Dasari et al. 2018 Cancer 124, 807-815; McNamara et al. 2020, Endocr. Relat. Cancer 27, R219-R238; Frizziero et al. 2022, Clin. Cancer Res. 28, 1999-2019. While patients with well-differentiated NETs generally have a better prognosis than NECs, overall survival depends on NET stage and histology. For example, while 5-year survival probability is 35% in GEP-NET patients with well or moderately-differentiated distant metastases, it is only 4% in GEP-NET patients with poorly-differentiated distant metastases. See, e.g., Diez et al. 2013, Ann. Gastroenterol. 26, 29-36; Yao et al. 2008, J. Clin. Oncol. 26, 3063-3072.

Accordingly, in some embodiments, the methods of the present disclosures are directed to treating a neurocrine neoplasm (NEN) with any of the compositions, including in combination with any of the immunotherapies, as disclosed herein.

In embodiments, the NEN is a pulmonary NEN. In embodiments, the NEN is an extrapulmonary NEN, and, more particularly, is a gastroenteropancreatic NEN (GEP-NEN).

In embodiments, the NEN is a well-differentiated neuroendocrine tumor (“WD-NET” or “NET”). In embodiments, the NET is a low-grade (G1), intermediate-grade (G2), or high-grade (G3) NET. In embodiments, the NET is a high-grade (G3) NET.

In embodiments, the NEN is a poorly-differentiated neuroendocrine carcinoma (“PC-NEC” or “NEC”). In embodiments, the NEC is a pulmonary NEC, and more particularly, the pulmonary NEC is a small cell lung cancer (SCLC) or a large cell lung cancer (LCLC).

In embodiments, the NEC is an extra-pulmonary NEC (EP-NEC), and more particularly, the EP-NEC is a GEP-NEC, including a small cell type NEC (SC-NEC) or a large cell type NEC (LC-NEC). In embodiments, the EP-NEC (including a GEP-NEC) is an advanced EP-NEC (or GEP-NEC). In embodiments, the EP-NEC (including a GEP-NEC) is a metastatic EP-NEC (or GEP-NEC). In embodiments, the EP-NEC (including a GEP-NEC) is an advanced and metastatic EP-NEC (or GEP-NEC).

In embodiments, the EP-NEC (including a GEP-NEC) is a refractory and advanced EP-NEC (or GEP-NEC). In embodiments, the EP-NEC (including a GEP-NEC) is a refractory and metastatic EP-NEC (or GEP-NEC). In embodiments, the EP-NEC (including a GEP-NEC) is a refractory and advanced and metastatic EP-NEC (or GEP-NEC).

In embodiments, the NEN is a is a mixed neuroendocrine-non-neuroendocrine neoplasm (MiNEN). In embodiments, the MiNEN is a mixed neuroendocrine-non-neuroendocrine carcinoma. More particularly, the MiNEN may be a MiNEN with adenocarcinoma component, a colon MiNEN, a metastatic MiNEN, an advanced MiNEN, or a metastatic and advanced MiNEN.

In embodiments, the NEN, NET, or NEC can be categorized as Stage I. In any of the preceding embodiments, the NEN, NET, or NEC can be categorized as Stage II. In any of the preceding embodiments, the NEN, NET, or NEC can be categorized as Stage III. In any of the preceding embodiments, the NEN, NET, or NEC can be categorized as Stage IV.

### Patients

The treatment methods and compositions (which includes formulation) are intended for use in any patient in need thereof.

In embodiments, a patient has been diagnosed with cancer, and more particularly, neuroendocrine cancer.

In embodiments, the patient has previously received one or more lines of therapies for an NEN, more particularly, therapies for an NET, NEC, or MiNEN (including an MiNEN carcinoma). In embodiments, the patient has previously received one or more lines of therapies for an NEC, including a pulmonary NEC or an EP-NEC, and more particularly, an EP-NEC that is a GEP-NEC.

In embodiments, the patient has previously demonstrated intolerance to one or more lines of therapies for the NEN, more particularly, an NET, NEC, or MiNEN (including an MiNEN carcinoma). In embodiments, the patient has previously demonstrated tolerance to one or more lines of therapies for an NEC, including a pulmonary NEC or an EP-NEC, and more particularly, an EP-NEC that is a GEP-NEC.

In embodiments, the patient has previously received one or more lines of therapies for an NEN, more particularly, an NET, NEC, or MiNEN (including ab MiNEN carcinoma). In embodiments, the patient has previously received one or more lines of therapies for an NEC, including a pulmonary NEC or an EP-NEC, and more particularly, an EP-NEC that is a GEP-NEC.

In embodiments, the patient has previously progressed on a first-line therapy for an NEN, more particularly, an NET, NEC, or MiNEN (including an MiNEN carcinoma). In embodiments, the patient has progressed on a first-line therapy for an NEC, including a pulmonary NEC or an EP-NEC, and more particularly, an EP-NEC that is a GEP-NEC.

In embodiments, the patient is refractory to (e.g., has progressed on) one or more lines of therapies for the NEN, more particularly, an NET, NEC, or MiNEN (including an MiNEN carcinoma). In embodiments, the patient is refractory to (e.g., has progressed on) one or more lines of therapies for an NEC, including a pulmonary NEC or an EP-NEC, and more particularly, an EP-NEC that is a GEP-NEC.

In any of the preceding embodiments, a previous line of therapy comprises cytotoxic chemotherapy. In embodiments, the cytotoxic chemotherapy comprises a platinum-based drug. In embodiments, the cytotoxic chemotherapy comprises a platinum-based combination therapy, and more particularly, comprises a platinum-based drug and a topoisomerase inhibitor. In embodiments, the platinum-based combination therapy comprises cisplatin (or carboplatin) and etoposide. In embodiments, the cytotoxic chemotherapy comprises an alkylating agent-based chemotherapy, and more particularly, comprises a temozolomide-based regimen.

In embodiments, the patient has histologically or cytologically confirmed relapsed high-grade EP-NECA and has failed at least one standard line of therapy.

In embodiments, the patient has histologically or cytologically confirmed refractory unresectable high-grade EP-NECA and has failed at least one standard line of therapy.

In embodiments, the patient has histologically or cytologically confirmed unresectable advanced high-grade extra-pulmonary neuroendocrine carcinoma (EP-NECAs), and have failed at least one standard line of therapy.

In embodiments, the patient has histologically or cytologically confirmed unresectable metastatic high-grade EP-NEC and has failed at least one standard line of therapy.

In embodiments, the methods comprise treating a patient with an advanced or metastatic extra-pulmonary neuroendocrine carcinoma (EP-NEC) with a TQ composition as disclosed herein, such as NP-101 (TQ Formula), including in the oral dosage forms described herein.

In embodiments, the EP-NEC patient has a median duration of response of at least (or about): 5 months, 5.5 months, 6 months, 6.5 months, 7 months, 7.5 months, 8 months, 8.5 months, 9 months, 9.5 months, 10 months, 10.5 months, 11 months, 11.5 months, or 12 months.

In embodiments, the EP-NEC patient has a median progression free survival of at least (or about): 2 months, 3 months, 4 months, 5 months, 6 months, 7 months, 8 months, 9 months, 10 months, 11 months, or 12 months.

In embodiments, the EP-NEC patient has a mean progression free survival of at least (or about): 1 month, 2 months, 3 months, 4 months, 5 months, 6 months, 7 months, 8 months, 9 months, 10 months, 11 months, or 12 months.

In embodiments, the EP-NEC patient has a likelihood of a 12-month PFS of at least (or about): 20%, 30%, 40%, 50%, 60%, 70%, or 80%.

In embodiments, the EP-NEC patient has a median overall survival (OS) of at least (or about): 6 months, 7 months, 8 months, 9 months, 10 months, 11 months, 12, months, 13 months, 14 months, 15 months, 16 months, 17 months, 18 months, 19 months, or 20 months.

In embodiments, the EP-NEC patient has an OS of least (or about): 4 months, 5 months, 6 months, 7 months, 8 months, 9 months, 10 months, 11 months, 12, months, 13 months, 14 months, 15 months, 16 months, 17 months, 18 months, 19 months, or 20 months.

In embodiments, the EP-NEC patient is a responder to treatment and has an OS of at least (or about): 6 months, 7 months, 8 months, 9 months, 10 months, 11 months, 12 months, 13 months, 14 months, 15 months, 16 months, 17 months, 18 months, 19 months, or 20 months.

In embodiments, the EP-NEC patient is a responder to treatment and has a likelihood of a 12-month PFS of at least (or about): 20%, 30%, 40%, 50%, 60%, 70%, 80%, or 90%.

In embodiments, the EP-NEC patient shows a complete response to treatment. In embodiments, the EP-NEC patient shows a partial response to treatment. In embodiments, the EP-NEC patient shows stable disease.

In embodiments, the EP-NEC patient shows an increase in CD4 positive lymphocytes, such as helper T cells, in the blood during treatment. In embodiments, the EP-NEC patient shows an increase in CD8 positive lymphocytes, such as cytotoxic T cells, in the blood during treatment. In embodiments, the EP-NEC patient shows an increase in both CD4 and CD8 positive lymphocytes during treatment.

In embodiments, the EP-NEC patient may have one or more genetic markers or profiles, such as high TMB, MSI-H, mutant TP53, or loss of Rb1.

In embodiments, the EP-NEC patient has any combination of the preceding values, including efficacy measurements, immune cell indicators, and genetic markers. For example, in embodiments, the EP-NEC patient has a median duration of response to treatment of at least (or about) 7.5 months, has a median progression free survival (PFS) of at least (or about) 2 months, or has at least (or about) a 30% likelihood of a 12-month PFS. For example, in embodiments, the EP-NEC patient is a responder to treatment and has a likelihood of a 12-month PFS of at least (or about) 50%, 60%, or 70%; and %, and a likely 12 month survival of at least (or about) 50%, 60%, 70%, 80%, 90%, or 100%.

In embodiments, the EP-NEC patient is a GEP-NEC patient, and thus, the methods comprise treating a patient with an advanced or metastatic extra-pulmonary neuroendocrine carcinoma (GEP-NEC) with a TQ composition as disclosed herein, such as NP-101 (TQ Formula), including in the oral dosage forms described herein.

In embodiments, the GEP-NEC patient has a median duration of response of at least (or about): 6 months, 6.5 months, 7 months, 7.5 months, 8 months, 8.5 months, 9 months, 9.5 months, 10 months, 10.5 months, 11 months, 11.5 months, or 12 months.

In embodiments, the GEP-NEC patient has a median progression free survival of at least (or about): 2 months, 3 months, 4 months, 5 months, 6 months, 7 months, 8 months, 9 months, 10 months, 11 months, or 12 months.

In embodiments, the GEP-NEC patient has a mean progression free survival of at least (or about): 2 months, 3 months, 4 months, 5 months, 6 months, 7 months, 8 months, 9 months, 10 months, 11 months, or 12 months.

In embodiments, the GEP-NEC patient has a likelihood of a 12-month PFS of at least (or about): 30%, 40%, 50%, 60%, 70%, or 80%.

In embodiments, the GEP-NEC patient has a median overall survival (OS) of at least (or about): 6 months, 7 months, 8 months, 9 months, 10 months, 11 months, 12, months, 13 months, 14 months, 15 months, 16 months, 17 months, 18 months, 19 months, or 20 months.

In embodiments, the GEP-NEC patient has an OS of least (or about): 5 months, 6 months, 7 months, 8 months, 9 months, 10 months, 11 months, 12, months, 13 months, 14 months, 15 months, 16 months, 17 months, 18 months, 19 months, or 20 months.

In embodiments, the GEP-NEC patient is a responder to treatment and has an OS of at least (or about): 8 months, 9 months, 10 months, 11 months, 12 months, 13 months, 14 months, 15 months, 16 months, 17 months, 18 months, 19 months, or 20 months.

In embodiments, the GEP-NEC patient is a responder to treatment and has a likelihood of a 12-month PFS of at least (or about): 50%, 60%, 70%, 80%, 90%, or 100%.

In embodiments, the GEP-NEC patient shows a complete response to treatment. In embodiments, the GEP-NEC patient shows a partial response to treatment. In embodiments, the GEP-NEC patient shows stable disease.

In embodiments, the GEP-NEC patient shows an increase in CD4 positive lymphocytes, such as helper T cells, in the blood during treatment. In embodiments, the GEP-NEC patient shows an increase in CD8 positive lymphocytes, such as cytotoxic T cells, in the blood during treatment. In embodiments, the GEP-NEC patient shows an increase in both CD4 and CD8 positive lymphocytes during treatment.

In embodiments, the GEP-NEC patient may have one or more genetic markers or profiles, such as high TMB, MSI-H, mutant TP53, or loss of Rb1.

In embodiments, the GEP-NEC patient has any combination of the preceding values, including efficacy measurements, immune cell indicators, and genetic markers. For example, in embodiments, the GEP-NEC patient has a median duration of response to treatment of at least (or about) 13 months, has a median progression free survival (PFS) of at least (or about) 5.5 months; and has at least (or about) a 50% likelihood of a 12-month PFS. For example, in embodiments, the GEP-NEC patient is a responder to treatment and has a likelihood of a 12-month PFS of at least (or about) 60% or 70%, and a likely 12 month survival of at least (or about) 80%, 90%, or 100%.

### EXAMPLES

The following illustrative examples are representative of embodiments of the composition and methods described herein and are not meant to be limiting in any way.

### Human NEN Cell-Based Studies

### Example 1

### Anti-Tumor Activity of TQ in Human Neuroendocrine Cancer Cells

### Introduction

Thymoquinone (TQ, C10H1202) is the main bioactive component of black seed (Nigella sativa), also known as black cumin, in the family Ranunculaceae. Studies have suggested numerous effects for this herb, supporting its potential use in treating various disorders, including cancer. For example, human cell-based studies suggest antioxidant and anti-inflammatory properties. See, e.g. Bordoni 2019, Antioxidants 8, 51; Mahmoud and Abdelrazek, 2018, Biomed. Pharmacother. 115, 108783 . . . . Other in vitro studies support anti-proliferative and apoptotic effects of TQ in numerous cell lines, including colon, breast, brain, pancreatic, ovarian, larynx, colon, myeloblastic leukemia, osteosarcoma, and lung cancer cell lines. Shoieb et al. 2003, Int. J. Oncol. 22, 107-113; Gali-Muhtasib et al. 2004, Int. J. Oncol. 25, 857-866; Adinew et al. 2022, Nutrients 14, 2120; Banerjee et al. 2010, Nutr. Cancer 62, 938-946; Mohamed et al. 2022, Curr. Oncol. 29, 9018-9030 (Table 1); for review, see Imran et al. 2018, Biomed Pharmacother. 106, 390-402.

Such studies, however, have not addressed whether TQ compositions could have beneficial effects against neuroendocrine cancers, including high grade neuroendocrine cancers. To assess this possibility, the present study evaluated the activity of TQ in cellular models of human neuroendocrine cancer.

### Methods

### NE Cancer Cell Lines:

TQ activity was evaluated in two human pancreatic neuroendocrine tumor (pNEN) cell lines: (1) BON-1 cells, established from a lymph node metastasis of a pNET (Evers et al. 1991, Gastroenterology. 101, 303-311); and (2) QGP-1 cells, established from primary somatostatin producing pNET (Kaku et al. 1980, Gan. 71, 596-601). Whole exome characterizations of the BON-1 and QGP-1 cell lines have revealed mutations in NRAS and KRAS, respectively, implicating aberrations in the RAS pathway in both cell lines. See Vandamme et al. 2015, J. Mol. Endocrinol. 54, 137-147.

### Cell Viability Assay:

About 5000 Bon-1 or 5000 QGP-1 cells were grown in 96-well plates overnight. On the following day, the cells were exposed to the indicated concentrations of TQ for 72 hrs. At the end of the treatment period, a standard MTT assay was performed to measure cell proliferation and conversely, in the presence of TQ, the reduction in cell viability The values represent 6 replicates per dose from three independent experiments.

### Colony Formation Assay:

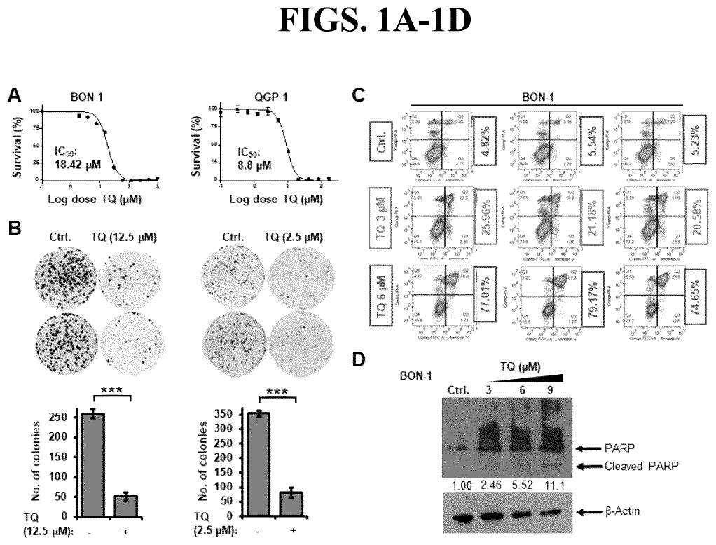

About 500 Bon-1 (or QGP-1) cells were seeded in 6-well plates in triplicate and allowed to attach overnight. On the following day, cells were treated with 2.5 uM or 12.5 uM TQ (or control) for 72 hrs. After treatment, cells were replenished with drug free media for 12 days and subjected to 0.5% crystal violet staining. Bar diagrams (FIG. 1B) show the number of colonies at the indicated TQ concentration.

### Apoptosis Assays:

About 30,000 BON-1 cells were grown in triplicate in 6 well plates overnight. The next day cells were exposed to 3 uM or 6 uM TQ (or control) for 72 h followed by Annexin V FITC apoptosis analysis. See, e.g., Demchenko 2012, Exp. Oncol. 34, 263-268. In addition, BON-1 cells grown and treated under similar experimental conditions were evaluated by Western blotting for the presence of cleaved PARP-a marker of apoptosis. See, e.g., Chaitanya et al. 2010, Cell Comm. Signal. 8, 31.

### Results

As shown in FIG. 1A, two pNEN cell lines were each acutely sensitive to TQ, with IC₅₀s in the low uM range. (In contrast, inhibitory action in a pancreatic ductal adenocarcinoma tumor models corresponded to TQ IC50s in the high uM range). Consistent with these observations, TQ also suppressed long term growth in a colony formation assay at similar low uM concentrations (FIG. 1B p<0.01).

Apoptosis studies corroborated these findings. TQ induced significant apoptosis of BON-1 cells in a dose-dependent manner when assayed by Annexin V-FITC staining and flow cytometry, as shown in FIG. 1C. As further corroboration, western blot analyses revealed a dose dependent accumulation of the PARP cleavage product-a marker of apoptosis. (FIG. 1D).

Collectively, these results indicate that NENs are sensitive to TQ. Strikingly, they also suggest that such neuroendocrine cancers may be particularly sensitive to TQ. The observed inhibition seen in the NEN cellular model was observed at lower TQ concentrations compared to inhibitory concentrations seen for other tumor models.

### Example 2

### Anti-Tumor Activity of TQ Plus ICPIs in Human Neuroendocrine Cells

### Introduction

NENs are highly vascular neoplasms, and angiogenesis is critically important for their growth and proliferation. Oberg et al. 2013, Clin. Cancer Res. 19, 2842-2849; Kuiper et al. 2011, World. J. Gastroenterol. 17, 219-225. NENs frequently express elevated levels of vascular endothelial growth factor (VEGF) ligand and its receptors, which can promote angiogenesis and tumor progression. Pavel et al. 2005, Clin. Endocrinol. 62, 434-443. Hypoxia and activated AKT and ERK signaling pathways play major roles in VEGF secretion and angiogenesis in NENs. Trisciuoglio et al. 2005, Mol. Biol. Cell 16, 4153-4162. AKT, also named protein kinase B (PKB), is a critical regulator generally involved in cell cycle and proliferation, and extracellular signal-regulated kinase (ERK) is a principal factor in mediating cell proliferation and survival. Xu et al. 2012, J. Oncol. 2012, 951724; Mebratu and Tesfaigzi 2009, Cell Cycle 8, 1168-1175.

Prior studies have suggested that TQ can inhibit tumor angiogenesis and tumor growth by suppressing AKT and extracellular signal-regulated kinase signal transduction pathways. See, e.g., Yi et al. 2008, Mol. Cancer Ther. 7, 1789-1796. Accordingly, TQ-mediated inhibition of AKT and ERK activation in endothelial cells could significantly impact angiogenesis pathways and increase the effectiveness of immunotherapy through its antiangiogenic and immunomodulation mechanisms. See Song and Finley 2020, Cell Commun. Signal. 18, 114; Murphy et al. 2006, Am. J. Pathol. 169, 1875-1885.

To assess whether TQ could enhance immunotherapy, the present study evaluated the anti-tumor efficacy of TQ plus dual immune checkpoint inhibitors: ipilimumab and nivolumab (Ipi-Nivo) in the pNEN cell line BON-1.

### Methods

BON-1 cells were exposed to the indicated concentrations of TQ or Ipi-Nivo or a combination of all three for 72 h followed by the standard MTT assay for cellular metabolic activity. Combination indices (CIs) were calculated using CalcuSyn Software. CIs less than 1 indicated a synergistic effect.

### Results

As shown in Table 1, the combination of TQ plus Ipi-Nivo was more effective in inhibiting Bon-1 cell line compared to single agent TQ (CI<1) at low doses. These results support therapeutic efficacy of TQ in combination with immune checkpoint inhibitors in treating pNENs.

TABLE 1
CI for Experimental Values

| | | Fraction | |
|---|---|---|---|
| TQ | Ipi + Nivo | Affected | |
| (uM) | (ug/ml) | (Fₐ) | CI |
| 15 | 25 | 0.35123 | 0.739 |
| 20 | 50 | 0.354323 | 0.987 |
| 25 | 100 | 0.463925 | 1.04 |

### Example 3

### Synergistic Activity of TQ Formula with a Single ICPI (Nivolumab) in Human Neuroendocrine Cancer Cells

Given the results in Example 2, additional experiments were conducted in pancreatic NEN cell lines to evaluate potential synergic effects of TQ Formula (NP-101) in combination with an immune checkpoint inhibitor, the anti-PD-1 antibody, nivolumab.

### Methods

NP-101 (BSO containing minimum standardized amounts of TQ) was first mixed with DMSO at a 1:5 ratio to prepare a stock solution which was used to prepare different dilutions with cell culture media for treatment of pNEN cells. The amount of TQ in each NP-101 dose was based on its 1.53% concentration in the TQ oil. Six TQ doses were evaluated in this experiment—30.6 μg/ml (1:500), 15.3 μg/ml (1:1000), 7.65 μg/ml (1:2000), 3.83 μg/ml (1:4000), 1.91 μg/ml (1:8000) and 0.96 μg/ml 1:1600). Similarly, six concentrations of nivolumab were evaluated-100 μg/ml, 50 μg/ml, 25 μg/ml, 12.5 μg/ml. 6.25 μg/ml, and 3.125 μg/ml. Combination indices (CIs) were calculated using CalcuSyn Software. Cells were evaluated with TQ alone, nivolumab alone, and TQ±nivolumab.

### Results

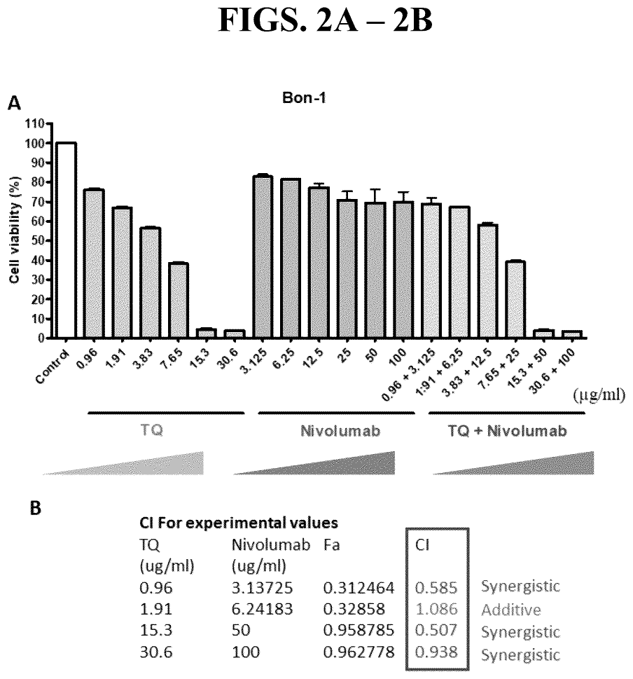

As shown in FIGS. 2A and 2B, NP-101 can suppress pNEN cell proliferation in a synergistic manner with nivolumab. At most of the doses evaluated, the combination index was less than 1, indicating a synergistic effect.

### Animal Studies

### Example 4

### Anti-Tumor Activity of NP-101 in Animal Model of Neuroendocrine Cancer

The previous studies demonstrated in vitro anti-tumor activity of NP-101 (TQ Formula) alone and synergistic activity in combination with IPI-Nivo. To explore potential in vivo effects, the anti-tumor activity of NP-101 (BSO containing minimum standardized amounts of TQ) was evaluated in a xenograft animal model of NEN cancer.

### Methods

BON-1 tumor fragments were implanted subcutaneously into ICR-SCID mice. After 1 week, NP-101 (200 ul BID) was administered twice daily, Monday-Friday, for 3 weeks, and mouse weight was recorded every 3 days.

### Results

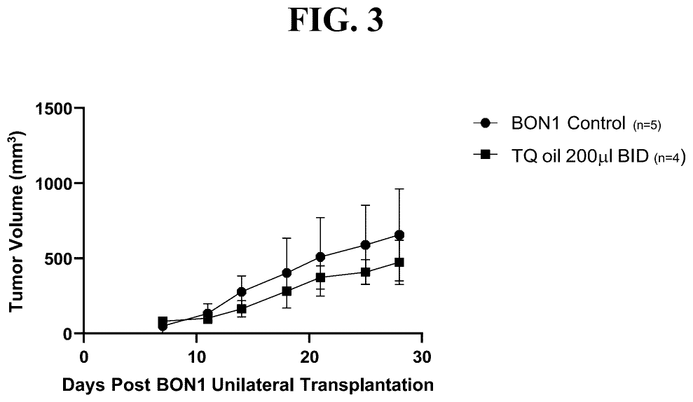

As shown in FIG. 3, oral administration of NP-101 (TQ Formula) resulted in considerable shrinkage of BON-1 pancreatic neuroendocrine tumors. Comparable results were seen in ICR-SCID mice receiving subcutaneous implants of QCP-1 tumor fragments. The treatment did not cause body weight loss and was well tolerated by the mice, with mild diarrhea noted towards the end of second week.

### Example 5

Anti-Tumor Activity of NP-101 in Animal Model of Hepatocellular Carcinoma To assess the potential efficacy of NP-101 (TQ Formula) against cancers other than pNENs, NP-101 was evaluated in a humanized mouse model carrying the hepatocellular cell line Hepg2 derived xenograft.

### Methods

Hepg2 cells were subcutaneously inoculated into humanized HuNog mice. NP-101 was administered orally (200 μL) 4 times per week, and tumor volumes were determined thereafter.

### Results

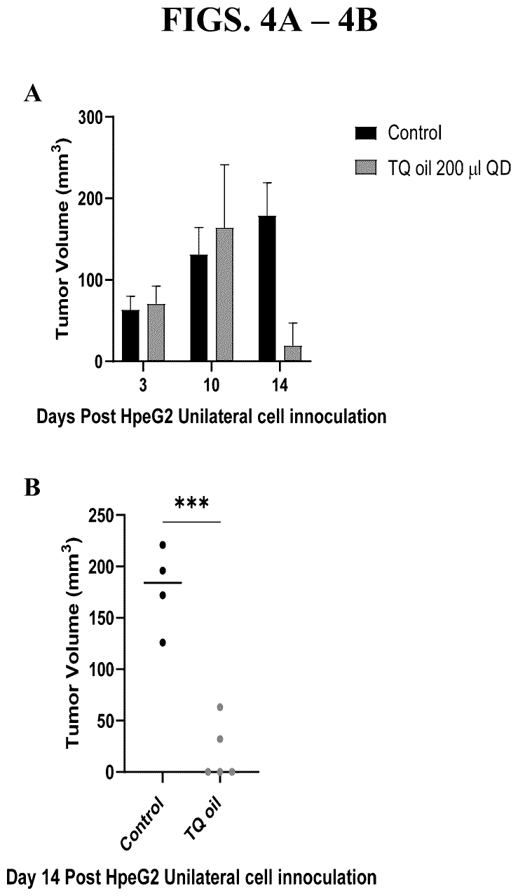

Oral administration of NP-101 (BSO containing minimum standardized amounts of TQ) lead to remarkable shrinkage of the HepG2 xenograft (FIG. 4A), revealing a statistically significant inhibition of tumor growth (p<0.0002) at day 14. (FIG. 4B) As with the Bon-1 xenograft, there was no outward toxicity associated with the treatment and dosing schedule indicating that NP-101 is well tolerated by the mice. These results indicate that NP-101 may have anti-tumor activity against carcinomas generally.

### Example 6

### Interaction of TQ Formula Components with PD-L1 and SHP2

### Background

The preceding experiments suggest that TQ components may play immunoregulatory roles in modulating anti-cancer response. To explore the molecular basis for such regulation, bioinformatic molecular modeling and docking experiments were conducted to determine whether TQ components, or related ingredients, might specifically interact with immune checkpoint proteins, including PD-L1 and SHP2.

The immune system has a key role in controlling cancer. Tumor cells express cancer-specific antigens derived from genetic alterations, and as such are targeted by the immune cells. This response is often inefficient because tumors can actively suppress immunity. Tumeh et al., 2014, Nature 515, 568-571. One mechanism for such suppression is engaging inhibitory immune checkpoint proteins, including the programmed death receptor 1 (PD-1) and cytotoxic T lymphocyte antigen-4 (CTLA-4), which are negative regulators of T-cell immune function. CTLA-4 functions during the priming phase of T-cell activation, while PD-1 functions during the effector phase, predominantly within peripheral tissues. See Sharma et al. 2021, Cancer Discov. 11, 838-857. Keir et al. 2008, Annu. Rev. Immunol. 26, 677-704.

In the case of PD-1, which is expressed predominantly on the surface of activated T cells, the inhibitory signal is provided by its ligand, PD-L1, which is naturally expressed on antigen-presenting cells (APCs) and in a variety of tissues. Riella et al. 2012, Am. J. Transplant. 12, 2575-2587. Under normal physiological conditions, this interaction keeps T-cell responses in check. In contrast, T-cell responses are markedly enhanced in PD-L1 knockout mice. T. Latchman et al. 2004, Proc. Natl. Acad. Sci. U.S.A. 101, 10691-10696.

In pathological settings like cancer, tumor cells can overexpress PD-L1 or other ICPI molecules. Prolonged exposure to PD-L1 leads to exhaustion (loss of function) of antigen-specific effector T cells, allowing tumor cells to evade immune attacks. See, e.g., Sharpe et al. 2018, Nat. Rev. Immunol. 18, 153-167; Gong et al. 2018, J. Immunother. Cancer 6, 8; Ostrand-Rosenberg et al. 2014, J. Immunol. 193, 3835-3841; Wang et al. 2016, Onco. Targets Ther. 9, 5023-5039. Indeed, this can lead to histological settings in which tumor tissue is infiltrated by immune cells that recognize but cannot eradicate the cancer cells. Kim et al. 2007, Immunology. 121, 1-14; Vinay et al. 2015, Semin. Cancer Biol. 35, S185-S198.

SHP2 (Src homology-2 domain-containing protein tyrosine phosphatase-2) is a non-receptor protein tyrosine phosphatase involved in CTLA-4 signaling. More particularly, SHP2 utilizes an SH2 domain to bind with CTLA-4 through a specific YVKM motif. This binding induces a conformational change in the SH2 domain, making the active site of SHP2 more accessible on an adjacent domain. Consequently, SHP2 dissociates from CTLA-4, allowing another protein to take its place and become activated. The activated SHP2 then dephosphorylates various cytoplasmic proteins at their tyrosine residues. Therefore, inhibiting the active site of SHP2 indirectly inhibits CTLA-4. See Khoroshilov et al. 2022, FASEB J. 36 (S1), R3699.

By targeting immune checkpoint proteins, including PD-1, PD-L1, and CTLA-4, cancer immunotherapy has emerged as powerful approach for treating cancer. Mahoney et al. 2015, Nat. Rev. Drug Discov. 14, 561-584; Hoos 2016, Nat. Rev. Drug Discov. 15, 235-247; Hu and Li, 2020, Front. Chem. 8, 601. Therapies targeting immune checkpoints like PD-1/PD-L1 can rescue exhausted T-cells and have demonstrated anti-tumor immunity. Indeed, the FDA has approved more than seven monoclonal antibodies targeting this the PD-1/PD-L1 interactions, with more in clinical trials. These include the anti PD-1 antibodies nivolumab (Opdivo, Bristol-Myers Squibb) and pembrolizumab (Keytruda, Merck) and the anti-PD-L1 antibodies atezolizumab (Tecentriq, Genentech/Roche), durvalumab (Imfinzi, AstraZeneca) and avelumab (Bavencio, EMD Serono, Inc.). Sharma et al. 2021.

Given the activity of NP-101 activity in cellular and cancer models, this study explored whether specific ingredients in NP-101 were able to interact with ICPIs and related signaling proteins, such as SHP2. As discussed below, these studies revealed significant binding interactions of multiple BSO ingredients to PD-L1 as well as SHP2.

### Methods

### PD-L1:

Molecular modeling studies were used to investigate whether the primary constituents of NP-101 (namely thymoquinone, oleic acid, linoleic acid, and palmitic acid) had significant affinity for the PD-L1 binding site engaged by the potent inhibitor, BMS-200.

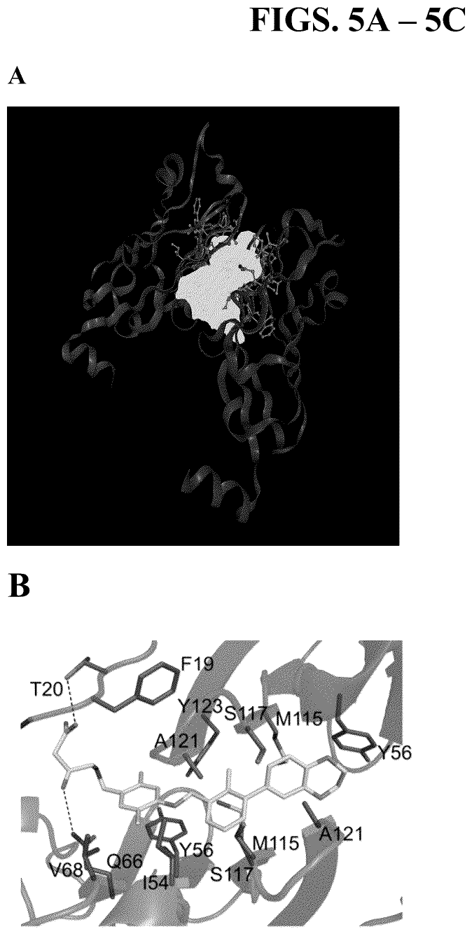

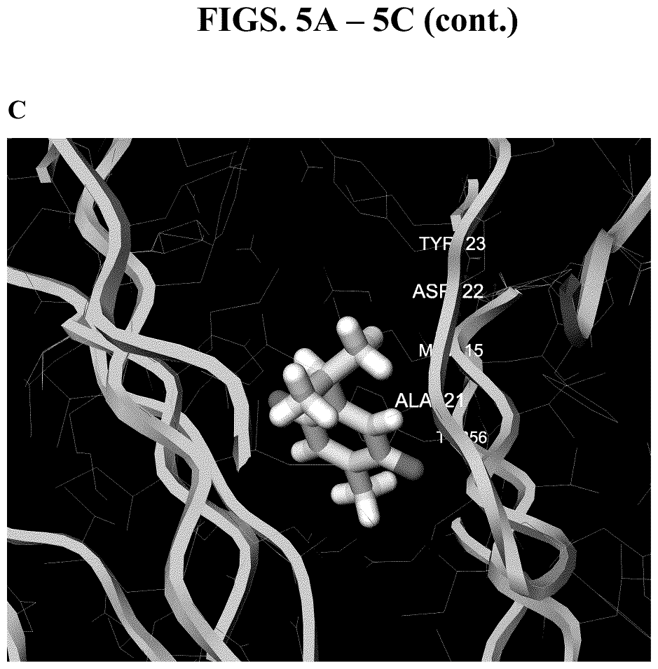

This was accomplished using the 5N2F model—the previously solved crystal structure for the PD-L1::BMS-200 complex. See Protein Data Bank entry 5N2F; Guzik et al. 2017, J. Med. Chem. 60, 5857-5867. As shown in FIG. 5A, this published structure reveals one inhibitor molecule at the center of a PD-L1 homodimer, filling a deep hydrophobic channel-like pocket between the two PD-L1 molecules. FIG. 5B is a higher resolution view of this binding site (as reported by Guzik 2017) revealing the interaction of BMS-200 with multiple PD-L1 residues, including Thr 20, Tyr 56, Gln 66, Met 115, Ala 121, Asp 122, and Tyr 123.

Selected NP-101 components were docked individually to the drug pocket within the 5N2F model, and their binding affinities were compared to the potent inhibitor BMS-200. These included thymoquinone and the fatty acids, oleic acid, linoleic acid, and palmitic acid. Lutterodt et al. 2010, LWT- Food Sci. Tech. 43, 1409-1413. This analysis was also carried out for Eicosapentaenoic acid (EPA) and Docosahexaenoic acid (DHA), two omega-3 fatty acids with antioxidant activity that have been reported in black seed oil. See, e.g., Kaskoos, 2011, Res. J. Med. Plants 5, 85-89; Hadjadj et al. 2014, Am. J. Food Technol., 9:136-143.

The results, presented in Table 2, show the comparative binding affinities for each component and the reference compound, BMS-200.

TABLE 2
PD-L1 Binding Affinities

| Compound | Description | Affinity |
|---|---|---|
| Positive Control | Reference compound (BMS-200) | −4.1 |
| Oleic Acid (OA) | BSO ingredient | −6.9 |
| Linoleic Acid (LA) | BSO ingredient | −6.9 |
| Palmitic Acid (PA) | BSO ingredient | −6.2 |
| Thymoquinone (TQ) | BSO ingredient | −7 |
| Eicosapentaenoic acid (EPA) | Omega-3 fatty acid | −7.6 |
| Docosahexaenoic acid (DHA) | Omega-3 fatty acid | −7.9 |

The results revealed that these NP-101 ingredients all showed higher binding affinity for the FN2F drug pocket (i.e. the values were more negative) than the reference compound, BMS-200. The omega-3 fatty acids EPA and DHA were most potent, followed by TQ, oleic acid, linoleic acid, and palmitic acid.

FIG. 5C depicts the interaction of TQ with the drug binding pocket of the PD-L1 dimer in the 5N2F complex. It shows close association of TQ with key residues of this pocket, including Tyr 56, Met 115, Ala 121, Asp 122, and Tyr 123.

### SHP-2:

Similar to the approach for PD-L1, potential binding of BSO components to SHP2 capitalized upon previous structural studies. Those studies showed the effects of a novel small-molecule inhibitor, 4,4′-(4′-carboxy)-4-nonyloxy-[1,1′-biphenyl]-3,5-diyl) dibutanoic acid (CNBDA), on SHP2 using a protein model called 4DGP (PDB: 4DG). See Protein Data Bank entry 4DGP; Hartman et al. 2020, ACS Omega, 5, 25113-25124. More particularly, those studies demonstrated that CNBDA, which targets the active site of SHP2, exhibited significant inhibitory effects against breast cancer cells.

Accordingly, the present study examined the potential impact of certain key ingredients in BSO on the same drug pocket targeted by CNBDA. Molecular docking techniques were used to compare the binding affinity of BSO ingredients compared to the reference molecule (CNBDA). The results, presented in Table 3, show the comparative binding affinities of each NP-101 component and the reference compound, BMS-200.

TABLE 3
SHP2 Binding Affinities

| Compound | Description | Affinity |
|---|---|---|
| Positive Control | Reference compound (CNBDA) | −4.5 |
| Oleic Acid (OA) | BSO ingredient | −4.4 |
| Linoleic Acid (LA) | BSO ingredient | −4.8 |
| Palmitic Acid (PA) | BSO ingredient | −4.0 |
| Thymoquinone (TQ) | BSO ingredient | −5.2 |
| Eicosapentaenoic acid (EPA) | Omega-3 fatty acid | −5.4 |
| Docosahexaenoic acid (DHA) | Omega-3 fatty acid | −5.9 |

The results revealed that most NP-101 ingredients assessed showed higher binding affinity for the 4DGP drug pocket (i.e. the values were more negative) than the reference compound, CNBDA. The omega-3 fatty acids, EPA and DHA, were most potent, followed by TQ and LA. OA and PA showed affinities lower that the reference compound.

### Discussion

In conclusion, molecular docking studies with both FN2F and 4DBG showed that ingredients from black seed oil, including LA, TQ, EPA, and DHA, showed higher binding activities compared to the reference compound. Among them, the omega-3 fatty acids EPA and DHA showed the highest affinity, followed by TQ, and then other fatty acids. Thus, these molecules offer promising candidates for further exploration in the context of disrupting PD-L1 ligand (or CTLA-4) activity and potentially treating cancers with high PD-1 and PD-L1 (or CTLA-4) expression levels.

Moreover, through their effects on PD-L1 or CTLA-4, these components may enhance or potentiate the efficacy of current dual ICPI therapies without the concomitant shortfalls of antibody therapies. While antibody therapies have greatly advanced options in cancer management, their efficacy varies depending on the malignancy and patient characteristics, and responses may diminish in the long-term. In addition, antibodies can be immunogenic, trigger adverse events, and even necessitate immune-suppressive therapy. Lipson et al. 2011, Clin Cancer Res. 17, 6958-6962; Dömling and Holak, 2014, Angew. Chem. Int. Ed. Engl. 53, 2286-2288; Powles et al. 201, Nature. 515, 558-562; Topalian et al. 2015, Cancer Cell. 27:450-461.

BSO and its components have an excellent safety profile, representing new therapeutic options that may enhance the efficacy and predictability of immunotherapy and offer better combinations with targeted therapies. More generally, BSO components reflect the benefits that small molecules offer over therapeutic antibodies, including lower production costs, higher stability, improved tumor penetration, amenability for oral administration and elimination of immunogenicity issues. Indeed, these advantages reflect a rising increase in the search for effective small molecules in immunotherapy. Zhan et al. 2016, Drug Discov Today. 21, 1027-36; Offringa et al. 2022, Nat. Rev. Drug Discov. 21, 821-840; Liang et al. 2023, Int. J. Mol. Sci. 24, 1280; Guzik et al. 2017, J. Med. Chem. 2017, 60, 5857-5867; Muszak et al. 2021, J. Med. Chem. 2021, 64, 11614-11636.

### Human Studies

### Example 7

### Human Clinical Studies: TQ Plus Immunotherapy in Treating Extrapulmonary Neuroendocrine Carcinoma

### Introduction

Neuroendocrine neoplasms (NENs) are a diverse group of cancers, whose annual incidence has increased over the last decade. Dasari et al. 2017. NENs can be divided into well-differentiated neuroendocrine tumors (NETs) and poorly differentiated neuroendocrine carcinomas (NECs) independent of the site where they arose. See, e.g., Nagtegaal et al. 2020, Histopathology 76, 182-188.

Given their poorly differentiated status, NECs are an aggressive subgroup with rising incidence and mortality. The majority of poorly differentiated NECs (˜90%) originate from the lung, namely, small cell lung cancer (SCLC; ˜86%) and large cell pulmonary neuroendocrine carcinoma (LCLC; ˜4%). See Dasari et al. 2018, Cancer 124, 807-815.

However, a significant fraction (˜10%) of poorly differentiated NECs arise from anatomical sites other than the lungs and accordingly, are termed extrapulmonary NECs (EP-NECs). Of such EP-NECs, about a third (˜37%) arise in the GEP tract, about a quarter (˜28%) are of unknown origin, while the others (˜34%) arise primarily in the head and neck and genitourinary tract (e.g., prostate, bladder, uterine, and cervix). Dasari et al. 2018.

Most patients with EP-NEC, regardless of their primary site, are initially diagnosed having metastatic disease with poor prognoses. Such high-grade NECs are aggressive tumors characterized by fast growing cells, and more than two-thirds of patients with GEP-NECs have distant metastases at the time of diagnosis. Sorbye et al. 2013, Ann. Oncol. 24, 152-160; Walter et al. 2017, Eur. J. Cancer 79, 158-165. Survival is poor, ranging from 38 months in patients with localized disease to 5 months in the metastatic setting, and it can be as short as 1 month in those receiving best supportive care alone in the metastatic setting. Sorbye et al. 2013; Sorbye et al. 2014, Cancer 120 2814-2823. Indeed, without chemotherapy, the median overall survival (OS) can be as short as 1 month. Moertel et al. 1991, Cancer 68, 227-232; Sorbye et al. 2014). Yet even with platinum/etoposide therapy, the median progression-free survival (PFS) and median OS in several multicenter studies were about 4-6 months and 7-16 months, respectively. See, e.g., Heetfeld et al 2015, Endocr. Relat. Cancer 22, 657-664; Yamaguchi et al. 2014, Cancer Sci. 105, 1176-1181; Celik et al. 2002, North Clin Istanb. 9, 35-40. In a population-based retrospective study in the Netherlands, which included 1544 cases (1045 with EP-NEC and 499 with NEC of unknown primary), the overall 5-year relative survival was 38% for patients with local/regional disease (n=447) and 7% for patients with extensive disease (n=582). For patients with NECs of unknown primary (n=499), the 5-year relative survival was 6%. Van der Zwan et al. 2018, Neuroendocrinology. 107, 50-55.

### Current Treatment Options

Reflecting these poor outcomes, treatment options of EP-NEC (and high grade NENs generally) are limited. In the first-line setting, the standard treatment for such patients is cytotoxic chemotherapy, typically systemic platinum-based chemotherapy. Dasari et al. 2018. Indeed, platinum/etoposide combination chemotherapy has been used as the first-line treatment since the 1990s, due to the histological similarity with small cell lung cancer and the presence of anti-tumor activity. Moertel et al. 1991; Terashima et al. 2012, Neuroendocrinology. 96, 324-332.

After progression on first-line chemotherapy, there are currently no standard of care options (and poor outcomes) for EP-NEC patients. Similar challenges face high grade GEP-NETs, which are significantly less responsive to platinum-based chemotherapy due to their distinct molecular pathogenesis and course of pathological grade transition. Sorbye et al. 2013; Frizziero et al. 2022. Genetic analyses have revealed that NECs usually have TP53 or Rb1 mutations, compared with NETs, which usually have mutations in MENI, DAXX and ATRX. In addition, VEGF and activation of PI3K-AKT and Raf-MEK-ERK pathways are implicated in the pathogenesis of NECs, as well as high-grade NET. While these findings have provided some diagnostic value and mechanistic insights, they have not yet led to effective therapies. Oberg et al. 2013, Clin. Cancer Res. 19, 2842-2849; Kuiper et al. 2011, World. J. Gastroenterol. 17, 219-225; Pavel at al. 2005, Clin. Endocrinol. 2005, 62, 434-443. Similarly, surgical resection cannot be performed in most EP-NEC cases due to the high rate of metastatic disease-up to 85%-at initial diagnosis. Garcia-Carbonero et al. 2016, Neuroendocrinology 103, 186-94.

Consequently, patients with NECs (and high-grade NETs) who have progressed or are intolerant or refractory to first-line chemotherapy regimens have limited treatment choices, due to lack of approved treatment of supportive literature for standard of care. Effective treatments for patients in second-line setting are therefore urgently needed.

### Immunotherapy

Cytotoxic T-lymphocyte-associated antigen 4 (CTLA-4) and the PD-1/PD-L1 interaction are immune checkpoint pathways that have emerged as targets for immunotherapy. Despite remarkable success of immune checkpoint inhibitors (ICPIs) in treating numerous non-neuroendocrine cancers to attain long-term survival, such immunotherapies have to date shown only limited benefits in treating high grade NENs. See, e.g., Xu et al. 2021, World J. Gastroenterol. 27, 8123-8137.

### Single ICPIs:

Recent NEC trials evaluating single-agent ICPIs in poorly differentiated NEC have shown disappointingly low response rates (3-5%) and poor outcomes (overall survival <6 months). See, e.g., Vijayvergia et al. 2020, Curr. Treat. Options Oncol. 21, 96. Vijayvergia et al. 2020, Br J Cancer 122, 1309-1314. Another small single arm study evaluated single agent pembrolizumab (anti-PD1) in patients with refractory high grade (G3). NENs showed low objective response rate (ORR) of 5% with poor outcome of overall survival of about 5 months (20.43 weeks) and median progression-free survival (PFS) of about 2 months (8.86 weeks). Vijayvergia, et al., 2020, Curr. Treat. Options Oncol. 21, 96. Another phase II study used single agent spartalizumab in refractory NEC and showed similarly low ORR (4.8%) and short PFS of 1.8 months and short OS of 6.8 months in a high-grade NEC group of 21 patients. Yao, et al. 2021, Endocr. Relat. Cancer 28, 161-172. In conclusion, monotherapy with single-agent ICPIs is not currently considered as a viable standard of care option for refractory EP-NEC due to its limited activity in these patients.

### Dual ICPIs:

Given the suboptimal efficacy of single agent ICPIs, the potential efficacy of dual checkpoint inhibitors has been evaluated in three recent phase II trials: DART SWOG 169 (Patel et al. 2020, Clin. Cancer Res. 26, 2290-2296; Patel et al. 2021, Cancer 127, 3194-3201); CA209-538 (Klein et al. 2020, Clin Cancer Res, 26, 4454-4459); and GCO-001 IPINEC (Girard et al. 2021, Ann. Ocol. 32 (suppl_5): S1283-S1346.) These studies explored the benefit of dual checkpoint inhibitors (anti-CTLA-4 and anti-PD-1 blockade) in treating high grade NENs, including poorly differentiated NECs. However, these studies have shown only limited improvement or benefits.

The DART SWOG 169 trial results were disclosed in two reports. The first report (Patel et al. 2020) suggested a benefit of the combination of nivolumab and ipilimumab in high-grade neoplasms. But it was a post-hoc analysis by grade (with microsatellite instability status not available for many patients) and did not distinguish between well-differentiated and poorly differentiated NENs. To address these limitations, the second report (Patel et al. 2021) focused on a dedicated prospective cohort of 19 patients with high-grade neuroendocrine neoplasms, including 11 with poorly differentiated NECs. However, these results revealed an ORR of only 26% (95% CI: 11-45%), a median PFS survival of 2.0 months, and a median OS of 8.9 months.

The CA209-538 trial (Klein et al. 2020) evaluated the potential of a combination of ipilimumab, using a different dosing regimen than the DART trial, and nivolumab in patients with advanced neuroendocrine tumors. In keeping with the DART trial findings, the CA209 report indicated a higher response rate to combination immunotherapy in patients with high-grade NENs, including pancreatic NETs and NECs. However, the ORR was only 24%, with clinical responses seen in only 2 of 10 NEC patients.

The GCO-001 IPINEC trial (Girard et al. 2021) compared single versus dual ICPI therapy in patients with poorly differentiated tumors. The results showed only limited improvements. This large trial, comprising about 50 centers, was a randomized (1:1) phase 2 study comparing nivolumab versus nivolumab plus ipilimumab in 185 patients with refractory (EP resistant) poorly differentiated NEC. The primary endpoint was objective response rate (ORR) at 8 weeks. Secondary endpoints included progression-free survival (PFS), overall survival (OS), NCT number: NCT03591731. The outcomes were poor: The ORR was 7.2% (versus 14.9%) in the single and dual ICPI groups, respectively. The PFS was 1.8 months (versus 1.9 months) in the single and dual ICPI groups, respectively. And OS was 7.2 months (95% CI [3.7-14.1]) in the single ICPI group and was even shorter-5.8 months (95% CI [3.3-7.6]) in the dual ICPI group.

Another recent phase II trial (DUNE) evaluated the activity and safety of dual checkpoint inhibitors based on anti-CTLA-4 (tremelimumab) and anti-PD-L1 (durvalumab) antibodies in advanced NENs, e.g., NENs that have spread, returned, or are unlikely to be cured. Capdevila et al. 2023, Nat. Commun. 14, 2973. Of the 123 patients in this study, 33 presented with high-grade (G3) GEP-NENs (including 18 GEP-NECs) and were assigned to Cohort 4. For this Cohort 4, the ORR was 9.1% (with responses limited to 3 NEC patients), the median OS was 5.9 months, and the median PFS was 2.4 months.

With their low survival outcomes and response rates, these ICPI trials underscore the complexities and difficulties in treating poorly differentiated endocrine cancers, challenges further exacerbated by the lack of predictive biomarkers to ICPIs. New innovative therapeutic approaches are urgently needed to improve immunotherapy efficacy in this highly recalcitrant group of EP-NEC patients, especially in the second line setting (after progression of platinum-based therapies) which lacks any approved therapies and presents a major clinical challenge given the dismal survival outcome of patients who may only survive for a few months.

The results from cellular, animal, and docking models in Examples 1-6 raised the intriguing possibility that TQ and BSO might be a useful adjunct to immunotherapy of these patients. Accordingly, the present clinical study explored the potential use of a new therapeutic regimen comprising TQ in combination with dual ICPI immunotherapy. As discussed in the case series below, four patients with metastatic poorly differentiated GEP-NEC administered BSO (black seed oil) capsules with dual immune checkpoint inhibitors (nivolumab plus ipilimumab) showed significantly improved response rates (>50% tumor reduction), progression free survival (PFS) and overall survival (OS).

### Methods

A set of patients at University Hospitals (UH) Seidman Cancer Center were treated with a combination of over-the-counter thymoquinone-based black seed oil (BSO) capsules (three capsules of 500 mg orally per day) at the time that they were being treated with dual ICPIs (nivolumab plus ipilimumab) in the second-line setting.

The patients were refractory to (and therefore had progressed) cytotoxic chemotherapy. These patient included three with metastatic poorly differentiated EP-NEC and one with mixed neuroendocrine-non-neuroendocrine neoplasm (MiNEN). The data were collected in accordance with an IRB-approved retrospective data analysis protocol at UH Seidman Cancer Center.

### Results

### Case 1

A 77-year-old male with a past medical history of anxiety and coronary artery disease, post-coronary artery bypass graft surgery (CABG) in 1991, was diagnosed with a poorly differentiated NEC of an unknown primary site. The diagnosis was formulated on biopsies of an axillary lymph node and liver mass. The patient was found to have left axillary lymphadenopathy on a screening lung CT scan. Biopsy showed a poorly differentiated NEC (Ki-67>90%). Immunohistochemical analysis showed that the tumor cells expressed chromogranin, CAM5.2, and CK20 but were negative for TdT and PAX5; the Ki67 was >90%. Next-generation sequencing (NGS) of the tumor confirmed TP53 and Tet 2 (TET2) mutation, the loss of Rb1, Microsatellite stable (MSS), and high tumor mutational burden (TMB: 53.2 m/MB). Skin mapping was performed, revealing no evidence of a Merkel cell. Anatomical scans with a CT and MRI of the abdomen and pelvis were performed to complete staging and reported extensive liver metastases, gastrohepatic, periportal, paraaortic lymphadenopathy, diffuse spinal (thoracic, lumbar), and pelvic osseous metastases consistent with metastatic NEC.

Therapy was initiated with systemic carboplatin and etoposide for two cycles complicated by fatigue and myelosuppression. Restaging scans after a second cycle showed disease progression, so second-line therapy was started with dual ICPIs in the form of nivolumab 240 mg intravenous (i.v.) every 2 weeks and ipilimumab 1 mg/kg i.v. every 6 weeks. The patient was interested in holistic medicine and started taking black seed oil capsules, 500 mg, three tablets daily (1500 mg) at the same time as the second-line therapy was started.

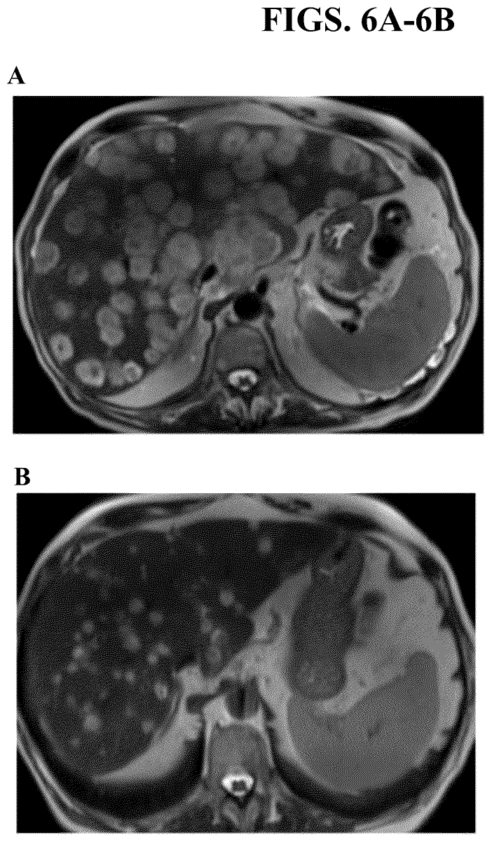

As shown in FIG. 6, a restaging scan after two cycles showed a significant response in liver lesions, lymph nodes, and osseous lesions (70% shrinkage per RECIST 1.1). The patient also had significant clinical improvement including weight gain of more than 10 pounds over a four-month period and improved energy levels.

The treatment was well tolerated with no significant side effects. The patient finished four cycles of nivolumab plus ipilimumab and thymoquinone BSO tablets, and a restaging scan after cycle 4 showed continuing partial response. This patient remained on maintenance treatment with nivolumab 240 mg i.v. every 2 weeks and TQ-based BSO tablets, and at 24 months, continued to show no side effects and an excellent quality of life, with subsequent restaging scans continuing to show partial response.

### Case 2

A 75-year-old man with a past medical history of chronic obstructive pulmonary disease (COPD), polymyalgia rheumatica (PMR), and hyperlipidemia was diagnosed with metastatic poorly differentiated NEC of the gall bladder, i.e. hepatobiliary origin. In June 2020, this patient presented to his primary care physician with complaints of abdominal pain, fatigue, and weight loss. An abdominal ultrasound identified an intrahepatic biliary mass; MRI confirmed the presence of a gall bladder mass that measured 8.5×10.3×6.2 cm and eroded into substantial portions of the posterior segment of the right hepatic lobe and involved a peripancreatic lymph node. A biopsy of the right liver mass was diagnosed as poorly differentiated NEC, small cell type. The malignant cells were immunohistochemically positive for cytokeratin CAM 5.2, synaptophysin, and CD56, and negative for cytokeratin AE1/3, chromogranin, and CD20. Ki-67 was 85%. NGS confirmed mutant PTEN, TP53, Rb1 intact, MSI-H, and high TMB (53.2 m/MB).

This patient started on carboplatin and etoposide and finished three cycles but could not tolerate the treatment due to worsening abdominal pain, nausea, vomiting, and weight loss. Restaging scans showed disease progression in both the primary tumor invading the right hepatic lobe and the peripancreatic lymph node.

Therapy was switched to dual ICPIs consisting of nivolumab 3 mg/kg i.v. and ipilimumab 1 mg/kg i.v. every 3 weeks as second-line therapy. The patient started taking BSO capsules, 500 mg, three tablets daily (1500 mg).

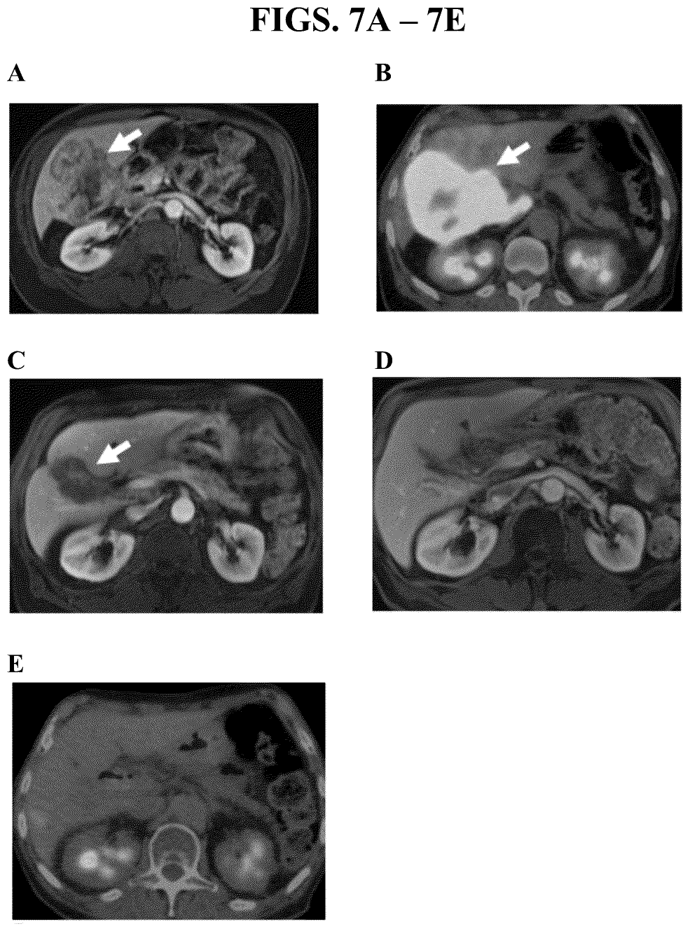

After two cycles of dual ICPI, restaging scans after two cycles of dual showed a significant response in both the primary tumor and the peripancreatic lymph node (60% shrinkage change per RECIST 1.1) (FIG. 7). The treatment was well-tolerated for a total of four cycles every 3 weeks with no significant side effects. The patient reported significant improvement in his energy levels, and he gained more than 10 pounds during the first 3 months of treatment. Restaging PET-CT scans demonstrated a complete response, and he is currently on maintenance therapy with nivolumab 240 mg i.v. every 2 weeks and TQ-based BSO tablets for 22 months with no evidence of new lesions.

### Case 3

A 67-year-old male with a past medical history of stage III B who has been diagnosed with Colon MiNEN (mixed neuroendocrine-nonneuroendocrine carcinoma with adenocarcinoma component). A restaging anatomical scan at that time excluded metastatic disease, and the patient had left laparoscopic hemicolectomy followed by adjuvant mFOLFOX chemotherapy. On surveillance, follow-up restaging scans showed new areas of metastatic disease in the liver, peritoneum, and abdominal lymph nodes, which were all FDG-avid on a PET/CT scan. Therefore, he underwent a liver biopsy, which confirmed metastatic poorly differentiated NEC. Immunohistochemical analysis showed that the tumor cells expressed chromogranin, synaptophysin, INSM-1, and CDX-2 consistent with NEC of gastrointestinal origin. Next-generation sequencing of the tumor confirmed TP53 mutation, the loss of Rb1, Microsatellite stable (MSS), and low TMB (6.3 m/MB). The patient started on carboplatin and etoposide and finished a total of six cycles. After three cycles, a restaging scan showed a partial response, but after cycle six scans, it showed disease progression in both liver and peritoneal metastases.

Therapy was switched to dual ICPIs, nivolumab 3 mg/kg i.v. and ipilimumab 1 mg/kg i.v. every 3 weeks; in addition, he began to ingest TQ-based BSO (one teaspoon of liquid form which is equivalent to 1500 mg oral tablets).

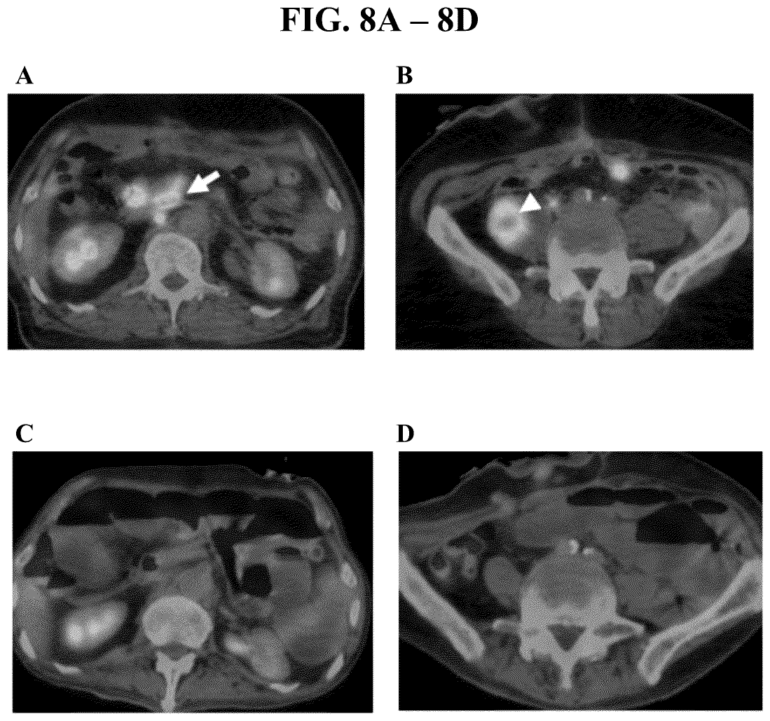

As shown in FIG. 8, after four cycles of immunotherapy and BSO, restaging scans demonstrated significant improvement in metastatic disease with decreased conspicuity of previously seen upper abdominal lymph nodes, and peritoneal nodules, and the liver lesions were no longer demonstrable. The patient had an overall complete response and was started on maintenance TQ-based BSO and nivolumab 240 mg i.v. every 2 weeks, with no evidence of progression yet (current PFS of 10 months).

### Case 4

An 85-year-old male with a past medical history of hypertension and coronary artery disease, CAD, BPH, and HTN, was diagnosed with a colon MiNEN (70% poorly differentiated NEC and 30% adenocarcinoma components). Staging CTs of the chest, abdomen, and pelvis excluded metastatic disease; therefore, he underwent laparoscopic right hemicolectomy; final pathology staging was T4N2bM0 with 50% poorly differentiated mixed small cell and large cell features (Ki67 95%) and a 50% adenocarcinoma component. NGS confirmed mutant BRAF, TP53, Rb1 loss, MSI-H, and high TMB (43.7 m/MB).

The patient refused platinum-etoposide as adjuvant therapy due to side effects, and instead, he agreed to be treated for 6 months with adjuvant mFOLFOX. After a 6-month course of adjuvant therapy with mFOLFOX, a restaging scan and PET-FDG showed disease progression with mainly an enlargement of the retroperitoneal, aortocaval, gastrohepatic, and right retrocrural lymph nodes. The therapy was switched to carboplatin and etoposide, and he finished six cycles with an initial good response after both cycle three and cycle six. However, further restaging imaging showed disease progression in less than six months from the last cycle of chemotherapy.

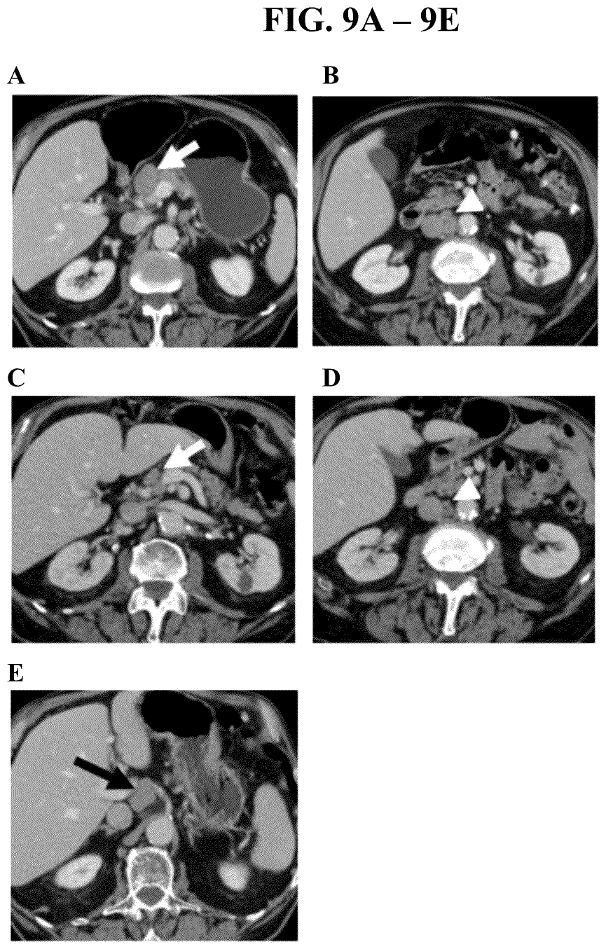

Therefore, he was switched to dual ICPIs, nivolumab 3 mg/kg i.v., and ipilimumab 1 mg/kg i.v. every 3 weeks. In addition, he began to ingest TQ-based BSO capsules, 500 mg, three tablets daily (1500 mg). As shown in FIG. 9, after completing two cycles of immunotherapy, restaging scans demonstrated significant improvement in retroperitoneal, aortocaval, and liver metastases. Repeated restaging scans after completing the three cycles continue to show treatment response with a significant decrease in the size of the retroperitoneal and aortocaval lymph nodes and no evidence of liver lesions. The patient maintained with no evidence of progression for 12 months, but he refused to resume maintenance therapy due to the development of immunotherapy-related pneumonitis. Subsequent restaging scans showed disease progression with new portocaval lymphadenopathy but he was not on any active therapy.

These results suggest that TQ compositions plus dual ICPIs may provide a promising therapeutic strategy for treating EP-NEC. After 4 cycles of BSO tablets plus dual ICPIs (nivolumab plus ipilimumab) combined therapy, restaging anatomical scans (CT and MRI) have demonstrated reduction in the size of both the primary and metastatic lesions (FIGS. 6-9). As summarized in Table 4, the four patients showed benefits with the combined regimen of BSO plus dual ICPIs with durable response and 3 out of 4 patients exceeded two years of PFS and OS. None of the four patients experienced significant toxicity, and all of them showed improvement in quality of life.

TABLE 4

| | | Patient | | | | | |
|---|---|---|---|---|---|---|---|
| | | Age at | | | | | |
| Primary | Histological | Diagnosis | | First Line | Second Line | Maintenance | |
| Tumor | Differentiation | (Years) | Gender | Treatment | Treatment | Therapy | Outcome |
| Unknown | EP Poorly | 77 | M | Carboplatin | Nivolumab 240 | Nivolumab 240 mg i.v. | Alive with PR and |
| Primary | differentiated | | | and Etoposide | mg intravenous | every 2 weeks, plus | PFS of 24 months |
| | neuroendocrine | | | | (i.v.) every 2 | BSO (TQ) tablets 1500 | |
| | carcinoma (Small | | | | weeks and | mg daily | |
| | Cell) | | | | Ipilimumab 1 | | |
| | | | | | mg/kg i.v. every | | |
| | | | | | 6 weeks for 4 | | |
| | | | | | cycles plus TQ- | | |
| | | | | | BSO (3 tablets | | |
| | | | | | 500 mg daily) | | |
| Gall | Poorly | 75 | M | Carboplatin | Nivolumab 3 | Nivolumab 240 mg i.v. | Alive with CR and |
| Bladder | differentiated | | | and Etoposide | mg/kg i.v. and | every 2 weeks, plus | PFS of 22 months |
| | neuroendocrine | | | | Ipilimumab 1 | BSO (TQ) tablets 1500 | |
| | carcinoma (small | | | | mg/kg i.v. every | mg daily | |
| | cell) | | | | 3 weeks for 4 | | |
| | | | | | cycles plus TQ- | | |
| | | | | | BSO (3 tablets | | |
| | | | | | 500 mg daily) | | |
| Colon | Mixed | 67 | M | Carboplatin | Nivolumab 3 | Nivolumab 240 mg i.v. | Alive with CR and |
| MiNEN | neuroendocrine- | | | and Etoposide | mg/kg i.v. and | every 2 weeks, plus | PFS of 10 months |
| | non- | | | | Ipilimumab 1 | BSO (TQ) tablets 1500 | |
| | neuroendocrine | | | | mg/kg i.v. every | mg daily | |
| | neoplasm | | | | 3 weeks for 4 | | |
| | (MiNEN) | | | | cycles plus TQ- | | |
| | | | | | BSO (1 teaspoon | | |
| | | | | | oil formula | | |
| | | | | | daily) | | |
| Colon | Mixed | 85 | M | Adjuvant | Nivolumab 3 | BSO (TQ) tablets 1500 | Alive with PR and |
| MiNEN | neuroendocrine- | | | mFOLFOX | mg/kg i.v. and | mg daily | PFS of 12 months |
| | non- | | | | Ipilimumab 1 | | |
| | neuroendocrine | | | | mg/kg i.v. every | | |
| | neoplasm | | | | 3 weeks for 3 | | |
| | (MiNEN) 70% | | | | cycles plus TQ- | | |
| | small cell and | | | | BSO (3 tablets | | |
| | 30% | | | | 500 mg daily | | |
| | adenocarcinoma | | | | | | |

M: male;
CR: complete response;
PR: partial response;
EP: extrapulmonary;
mFOLFOX: modified FOLFOX

Previous studies, as well as those described in Examples 1-6, suggest multiple potential mechanisms by which TQ may enhance the efficacy of ICPI therapy in treating EP-NEC patients, including anti-proliferative, apoptotic, immunoregulatory, anti-inflammatory, and antiangiogenic actions. Consistent with this combinatorial perspective, other clinical studies in different cancers have shown synergistic effect of anti-angiogenic therapies in combination with immune checkpoint inhibitors (ICPIs). Fukumura et al. 2018, Nat. Rev. Clin. Oncol. 5, 325340; Taylor et al. 2020, J. Clin. Oncol. 38, 1154-1163.

Thus, therapeutic regiments based on combinations of TQ compositions and ICPIs may offer a promising avenue for treating neuroendocrine cancers, including EP-NECs and other high-grade neuroendocrine tumors.

### Example 8

### Human Clinical Studies: NP-101 Plus Immunotherapy in Treating High-Grade Neuroendocrine Carcinoma

The purpose of this pilot clinical study was to evaluate the potential benefit of NP-101 (also referred to as TQ Formula), i.e., BSO containing minimum standardized amounts of TQ in a hardshell capsule, in combination with dual ICPIs (nivolumab and ipilimumab) in the second-line setting for EP-NECA. TQ Formula (TQ, C10H1202), provided in the form of black seed oil, is an investigational (experimental) drug that may enhance the effect that immunotherapy drugs such as ipilimumab and nivolumab (Ipi-Nivo) have on neuroendocrine carcinoma.

### Intervention:

Drug: NP-101 (TQ Formula; Black seed oil capsules): Oral, five 600 mg capsules daily every three weeks for four cycles (21-day cycle), then maintenance for an additional 12 weeks for a total of six cycles.

Drug: Nivolumab (Opdivo) 3 mg/kg): Intravenously on Day 1 every (21-day) cycle for four cycles (maximum dose 360 mg once every 3 weeks), then 240 mg maintenance every two weeks for six cycles for a total of six months of treatment.

Drug: Ipilimumab (Yervoy) (1 mg/kg): Intravenously on day 1 of each (21-day) cycle for a total of four cycles only.

### Inclusion and Exclusion Criteria:

Enrolled subjects were required to have histologically or cytologically confirmed relapsed high-grade extra-pulmonary neuroendocrine carcinoma and/or refractory unresectable advanced high-grade extra-pulmonary neuroendocrine carcinoma and/or metastatic high-grade extra-pulmonary neuroendocrine carcinoma (EP-NECAs).

In addition, they must have progressed on at least first line standard therapy for this disease. Subjects must have recovered from acute toxicityemotherapy. Any prior non-hematologic vital organ toxicity (cardiac, pulmonary, hepatic, and renal) of previous therapy must have resolved to grade 1 or less. Neurological toxicities must have resolved to grade 2 or less.

Subjects with well-differentiated GEP-NETs were excluded from treatment in this study.

### Primary Outcome Measures:

Primary outcome measure includes antitumor activity of NP-101 (TQ Formula) plus nivolumab and ipilimumab in subjects with advanced and/or metastatic EP-NECAs who progressed on first line therapy. Antitumor activity will be measured by the number of participants that have a complete response (CR), partial response (PR), or stable disease (SD). Time-Frame: Up to 6 months from the start of treatment.

CR is defined as the complete disappearance of all target lesions, confirmed by repeat assessments at no less than 4 weeks after the criteria for response is first met. PR is defined as at least a 30% decrease in the sum of diameters of target lesions, taking as reference the baseline sum longest diameter. SD is defined as neither sufficient decrease to qualify for a partial response nor sufficient increase to qualify for progressive disease.

### Secondary Outcome Measures:

Secondary outcome measure includes safety profile/toxicity of the combined administration of NP-101 (TQ Formula) with nivolumab and ipilimumab. More particularly, this measure is based on the number of participants who experience a Grade 3 or greater drug-related adverse events. Time-Frame: Up to 2 years from the start of treatment.

Secondary outcome measures also include time to progression (TTP) This is based on determining the time to progression (TTP) using NP-101 (TQ Formula) plus nivolumab and ipilimumab combined regimen in subjects with EP-NECAs. Time-Frame: Up to 6 months from the start of treatment.]

### Initial Results

The first five patients in an ongoing pilot study received NP-101 plus immunotherapy (Ipi-Nivo). Four of the five patients (80%) have responded. Of the four responders, three patients tolerated NP-101 well with no additional toxicities, showed improved quality of life and partial responses, with PFS now exceeding 6 months. The fourth responder had a PFS of 11 weeks but stopped NP-101 treatment for the last five weeks before imaging. All patients remain alive and in good health and therefore OS has not yet been determined.

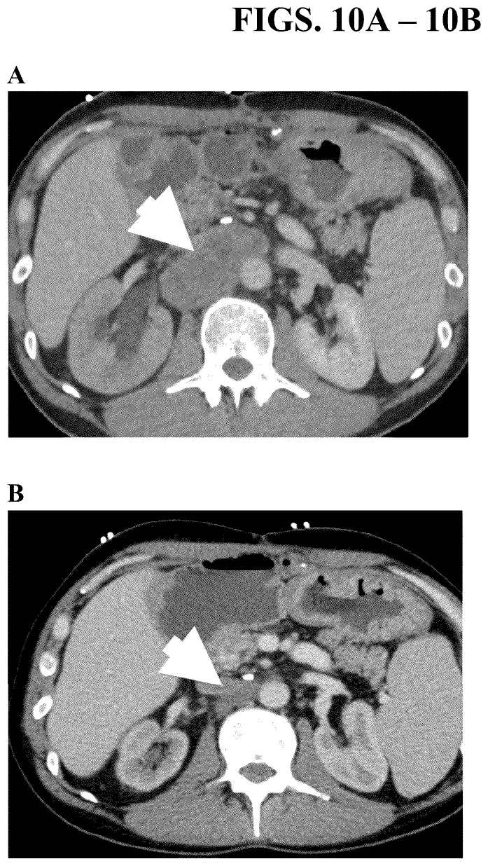

FIG. 10 shows abdominal CT scans in a patient (ID #4, Table 9) with metastatic refractory high grade colon NEC. The baseline scan (A) shows a large aortocaval nodal mass that was significantly reduced after four cycles of NP-101 plus Ipi-Nivo therapy (B).

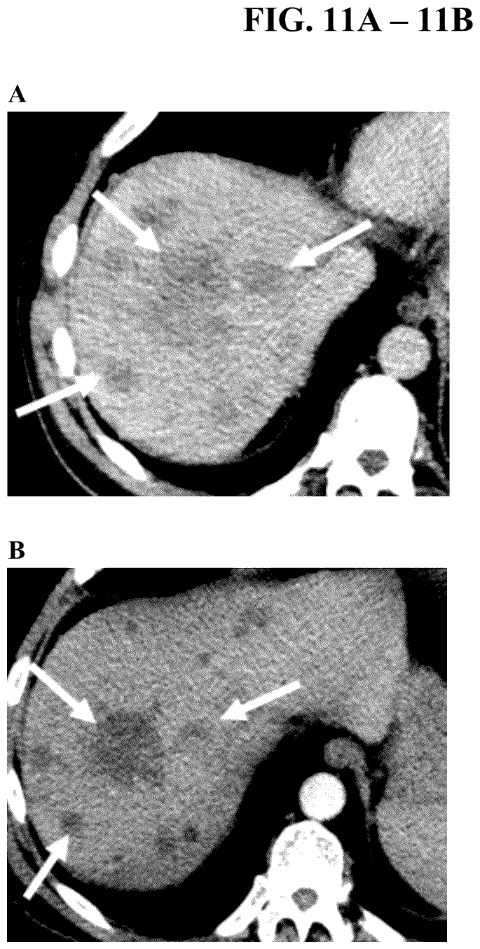

FIG. 11 shows abdominal CT scans in a second patient (ID #6, Table 9) with metastatic refractory high grade pancreas NEC. The baseline scan (A) shows multiple liver metastases (arrows) that decreased in size after just two cycles of NP-101 plus Ipi-Nivo therapy (B).

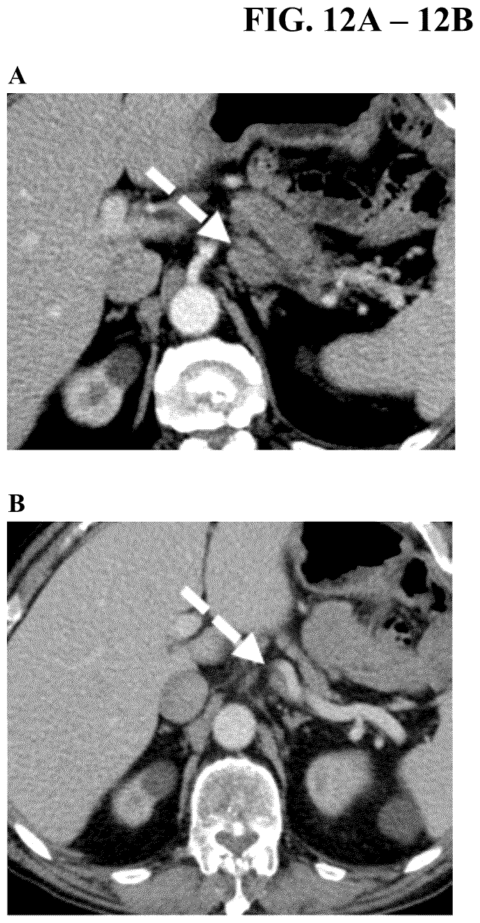

FIG. 12 shows abdominal CT scans in a third patient (ID #3, Table 9) with refractory high grade colon NEC. The baseline scan (A) shows a peripancreatic node that decreased in size after 4 cycles of NP-101 plus Ipi-Nivo therapy (B).

In sum, the use of the NP-101 drug substance, which includes a standardized concentration of TQ, represents a promising adjunct to immunotherapy. As shown here, NP-101 is a safe and well tolerated composition and may enhance outcomes in patients with high grade neuroendocrine cancer, including patients with neuroendocrine tumors and neuroendocrine carcinomas.

Notably, at least three patients responding to the NP-101 plus Ipi-Nivo ICPI immunotherapy were negative for PD-L1 expression. This observation highlights the possibility that NP-101 may modulate the immune environment in these EP-NEC patients to overcome resistance or enhance sensitivity to ICPI therapy. Indeed, 75% of the subjects (3 of 4) showed responses to the combination of NP-101 plus Ipi-Nivo, compared to the low response rates (e.g., 25% or less) and overall survival outcomes (e.g., 9 months or less) historically observed with Ipi-Nivo therapy alone. However, these results do not preclude the possibility that the NP-101 will also enhance the efficacy of ICPIs (e.g., Ipi-Nivo) in patients who are positive for expression of PD-L1. Indeed, ipilimumab targets the receptor (PD-1) for the PD-L1 ligand. This supports the use of the NP-101 plus Ip-Nivo combination in treating NECs in both PD-L1 negative and PD-L1 positive patients. Bearing on this view are clinical observations that elevated PD-L1 expression is not necessarily associated with higher responses rates, particularly in the context of multiple drug therapies. See, e.g., Yi et al. 2018, Mol. Cancer 17, 129. In addition, the docking studies presented here (Example 5) support multiple roles for NP-101 components, including the potential inhibition of CTLA-4 (and SHP2).

### Cumulative Results

The pilot clinical study was completed with 12 patients, all refractory to first line platinum-etoposide therapy and all receiving at least one dose of NP-101 plus immunotherapy (Ipi-Nivo).

Table 5 presents the demographic and baseline clinical characteristics for the 12 patients.

TABLE 5
Patient Profiles and Baseline Clinical Characteristics

| Item | Treatment Group | |
|---|---|---|
| Gender (%) | | |
| Male | 8 | (66.7%) |
| Female | 4 | (33.3%) |
| Age (Range) | 66.6 | (43-84) |
| Primary Tumor Site | | |
| GEP | 8 | (66.7%) |
| Gynecological | 2 | (16.6%) |
| Head and Neck | 1 | (8.3%) |
| Unknown | 1 | (8.3%) |
| Morphology | | |
| Small Cell | 6 | (50%) |
| Large Cell | 3 | (25%) |
| Indeterminate | 3 | (25%) |
| Ki67 Index (Average and Range) | 83% | (64%-98%) |
| Previous Treatment | | |
| Platinum-Etoposide | 9 | (75%) |
| Platinum-Etoposide with durvalumab | 1 | (8.3%) |
| Platinum-Etoposide then pembrolizumab | 1 | (8.3%) |
| Platinum-Etoposide the FOLFOX | 1 | (8.3%) |

Table 6 presents the next generation sequencing (NGS) results for all 12 EP-NEC patients. A deficiency in the MMR (mismatch repair) system can lead to microsatellite instability MSI. MSI status can be assessed in tumor samples and categorized as high (MSI-H) if 30% or more of the loci show instability, low (MSI-L), or microsatellite stable (MSS) if none of the microsatellite markers show instability.

TABLE 6
NGS Profile for Selected Markers

| Assay | Treatment Group |
|---|---|
| TMB (1 = High; 2 = Low) | |
| High | 3 (25%) |
| Low | 9 (75%) |
| MMR (1 = MSI-H; 2 = MSS) | |
| MSI-H | 1 (8.3%) |
| MSS | 11 (91.7%) |
| TP53 (1 = Wild-Type; 2 = Mutant) | |
| Wild-Type | 5 (41.6%) |
| Mutant | 7 (58.4%) |
| Rb1 (1 = Intact; 2 = Loss) | |
| Intact | 7 (58.4%) |
| Loss | 5 (41.6%) |

Treatment was generally safe and well-tolerated by the patients. No Grade 5 treatment-related adverse events (AEs) or death related events occurred in the patients. Grade 3/4 AEs were seen in 11 of 12 patients, most frequently rash (25%), nausea (16.7%), vomiting (16.7%), and transaminitis (16.7%). Grade ½ AEs were seen in all patients, most frequently fatigue (75%), nausea (41.7%), pruritis (41.7%), vomiting (25%), and abdominal pain (25%).

Table 7 shows efficacy measures for all 12 EP-NEC patients, as well as the subset of 8 GEP-NEC patients:

TABLE 7
Treatment Efficacy of NP-100 plus Ipi-Nivo

| | EP-NEC | GEP-NEC |
|---|---|---|
| Measure | (N = 12) | (N = 8) |
| ORR (%) | 41.7%¹ | 50%² |
| Complete Response (%) | 16.7% | 25% |
| Partial Response (%) | 25% | 25% |
| Median Duration of Response | 7.5 m | 13.4 m |
| (months) | | |
| PFS (months) | 1.96 ± 0.22 m (mean) | 1.90 ± 0.20 m (mean) |
| | 1.94 m (median)³ | 5.52 m (median)⁴ |
| 12-month PFS (%) | 31 ± 13% | 50 ± 18% |
| Median OS (months) | Not yet reached | Not yet reached |
| | (Currently 6.6 m)⁵ | (Currently 7.0 m)⁶ |
| 12-month survival (%) | 52 ± 18 m | 56 ± 19 m |

ORR: Objective Response Rate; PFS: Progression Free Survival; OS: Overall Survival; m: month
¹95% Confidence Interval (CI): 15.2, 72.3.
²95% Confidence Interval (CI): 16.0, 84.0.
³95% Confidence Interval (CI): 1.22, 8.77.
⁴95% Confidence Interval (CI): 1.22, upper limit not yet estimable.
⁵Preliminary calculated value (range 1.6 - 20.9).
⁶Preliminary calculated value (range 1.6 - 20.9).

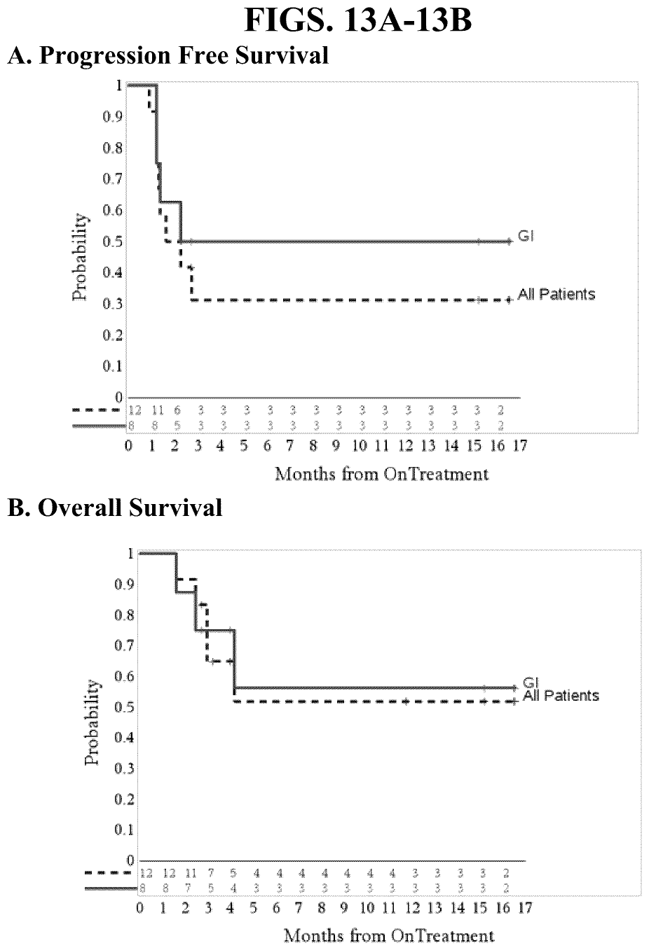

FIG. 13 shows the Kaplan-Meier PFS curve (A) and Kaplan-Meier OS curve (B) for all EP-NEC patients and the subset with GEP-NEC (GI). The data shows an upward shift of the PFS and OS curves for the GEP-NEC patient subset, compared to all EP-NEC patients.

Table 8 shows the 12-month PFS (and 12-month survival rate (SR)) for all patients comprising the EP-NEC cohort (12) and the subset of these patients (5) showing an objective response to treatment.

TABLE 8
Treatment Efficacy in All Patients and Responders

| Measure | All Patients (N = 12) | Responders (N = 5) |
|---|---|---|
| 12-month PFS (%) | 31 | 75 |
| 12-month SR (%) | 50 | 100 |

As shown in Table 9, these 5 responders were further evaluated by next generation sequencing (NGS) to evaluate tumor burden features and genetic loci.

TABLE 9
NGS Profile of Responders

| Patient | Primary | | | | | | Other |
|---|---|---|---|---|---|---|---|
| ID # | Tumor Site | Response | TMB | MMR | TP53 | Rb1 | Loci |
| 3 | Colon | CR | H-1 | MSI-H | Mutant | Intact | BRCA-1 |
| | | | | | | | CHEK-1 |
| 4 | Colon | CR | L | MSS | Wild-Type | Intact | |
| 6 | Pancreas | PR | L | MSS | Wild-Type | Intact | BRAF |
| | | | | | | | KRAS |
| | | | | | | | TET2 |
| 9 | Ovary | PR | L | MSS | Mutant | Loss | |
| 17 | Esophagus | PR | H | MSS | Mutant | Intact | |

As Table 8 shows, the responders included patients with high microsatellite instability (3), high tumor mutational burden (3, 17), mutant TP53 (3, 9, 17) and Rb1 loss (9).

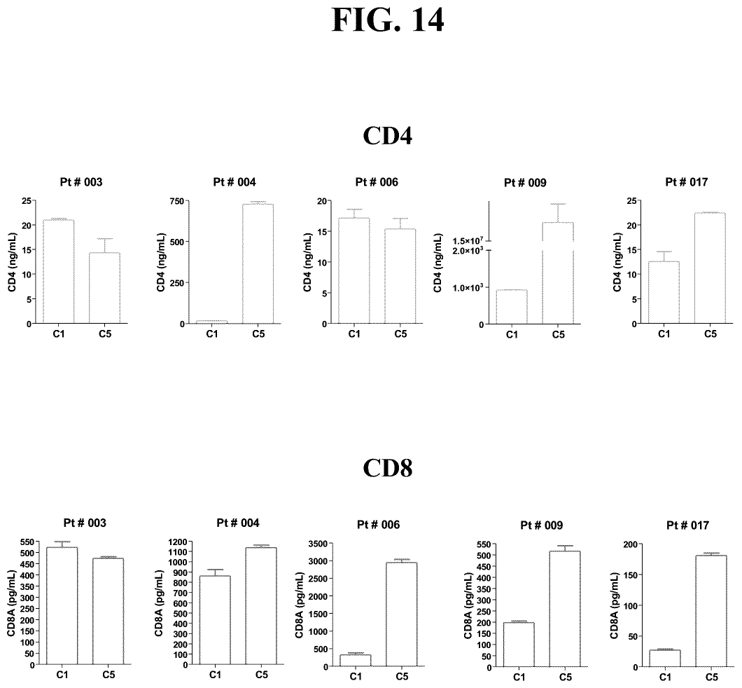

As shown in FIG. 14, EP-NEC responders 3, 9, 17 each showed an increase in CD4 positive T cells (helper T cells) and CD8 positive T cells (cytotoxic T cells) over the course of treatment. Responders 3 and 17 were GEP-NEC patients. T cell levels were measured from blood draws prior to first cycle (C1) and after the fourth cycle (C5) or

Together, these observations show that the combination of NP-101 plus dual ICPIs (nivolumab and ipilimumab) was safe and well-tolerated, with indicators of anti-neoplastic activity that was associated with enhancing CD4 and CD8 lymphocytes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

While this invention has been particularly shown and described with references to preferred embodiments thereof, it will be understood by those skilled in the art that various changes in form and details can be made therein without departing from the scope of the invention encompassed by the appended numbered embodiments. Further, all embodiments included herein are given solely for the purpose of illustration and are not to be construed as limitations of the present invention, as many variations thereof are possible without departing from the spirit and scope of the invention.

### Numbered Embodiments

The current disclosure is further directed to the following embodiments, which comprises part of specification.
1. A method for treating a neuroendocrine neoplasm (NEN), comprising administering to a patient in need thereof a therapeutically effective amount of a thymoquinone (TQ) composition in combination with an immunotherapy.
2. The method of embodiment 1, wherein the immunotherapy comprises administering a therapeutically effective amount of at least one immune checkpoint inhibitor.
3. The method of embodiment 2, wherein the immune checkpoint inhibitor is a PD-1 inhibitor, a PD-L1 inhibitor, or a CTLA-4 inhibitor.
4. The method of embodiment 3, wherein the immunotherapy comprises administering a therapeutically effective amount of a PD-1 inhibitor and a CLTA-4 inhibitor.
5. The method of embodiment 4, wherein the PD-1 inhibitor is an anti-PD-1 antibody and the CTLA-4 inhibitor is an anti-CLTA-4 antibody.
6. The method of embodiment 5, wherein the anti-PD-1 antibody is nivolumab (Opdivo®), pembrolizumab (Keytruda®), cemiplimab (Libtayo®), dostarlimab (Jemperli®), or retifanlimab (Zynx®).
7. The method of embodiment 5, wherein the anti-CTLA-4 antibody is ipilimumab (Yervoy®) or tremelimumab (Imjudo®).
8. The method of embodiment 3, wherein the PD-L1 inhibitor is an anti-PD-L1 antibody.
9. The method of embodiment 8, wherein the anti-PD-L1 antibody is atezolizumab (Tecentriq®), avelumab (Bavencio®), or durvalumab (Imfinzi®).
10. The method of embodiment 5, wherein the anti-PD-1 antibody is nivolumab and the anti-CTLA-4 antibody is ipilimumab.
11. The method of embodiment 10, wherein the nivolumab is administered on day 1 of a 3-week cycle for one or more cycles.
12. The method of embodiment 10, wherein the nivolumab is administered on day 1 of a 3-week cycle for four cycles and then administered on day 1 of a 2 week cycle for six cycles.
13. The method of embodiment 11 or 12, wherein the nivolumab is administered at a dose of about 3 mg/kg.
14. The method of embodiment 10, wherein the ipilimumab is administered on day 1 of a 3-week cycle for one or more cycles.
15. The method of embodiment 10, wherein the ipilimumab is administered on day 1 of a 3-week cycle for four cycles.
16. The method of embodiment 14 or 15, wherein the ipilimumab is administered at a dose of about 1 mg/kg.
17. The method of any one of embodiments 1-16, wherein the patient has previously received one or more lines of therapies for the NEN.
18. The method of any one of embodiments 1-16, wherein the patient has previously demonstrated intolerance to one or more lines of therapies for the NEN.
19. The method of any one of embodiments 1-16, wherein the patient has become refractory to or previously progressed on at least one line of cytotoxic chemotherapy.
20. The method of embodiment 19, wherein the cytotoxic chemotherapy comprises a platinum-based drug.
21. The method of embodiment 19, wherein the cytotoxic chemotherapy comprises a platinum-based combination therapy.
22. The method of embodiment 21, wherein the platinum-based combination therapy comprises a platinum-based drug and a topoisomerase inhibitor.
23. The method of embodiment 22, wherein the platinum-based combination therapy comprises cisplatin (or carboplatin) and etoposide.
24. The method of embodiment 19, wherein the cytotoxic chemotherapy comprises an alkylating agent-based chemotherapy.
25. The method of embodiment 24, wherein the alkylating agent-based chemotherapy is a temozolomide-based regimen.
26. The method of any one of embodiments 1-25, wherein the TQ composition comprises black seed oil (BSO).
27. The method of embodiment 26, wherein the BSO comprises 1.5-3.0 wt % TQ.
28. The method of embodiment 26, wherein the BSO comprises at least 1.5 wt %, at least 1.6 wt %, at least 1.7 wt %, at least 1.8 wt %, at least 1.9 wt %, at least 2.0 wt %, at least 2.1 wt %, at least 2.2 wt %, at least 2.3 wt %, at least 2.4 wt %, at least 2.5 wt %, at least 2.6 wt %, at least 2.7 wt %, at least 2.8 wt %, at least 2.9 wt %, or at least 3.0 wt % TQ.
29. The method of embodiment 26, wherein the BSO comprises about 1.5 wt %, about 1.6 wt %, about 1.7 wt %, about 1.8 wt %, about 1.9 wt %, about 2.0 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, or about 3.0 wt % TQ.
30. The method of embodiment 26, wherein the BSO comprises at least 1.5 wt % TQ or about 1.5 wt % TQ.
31. The method of embodiment 26, wherein the BSO comprises at least 1.7 wt % TQ or about 1.7 wt % TQ.
32. The method of any one of embodiments 26-31, wherein the BSO comprises 9-15 wt % palmitic acid (PA).
33. The method of embodiment 32, wherein the BSO comprises at least 9 wt %, at least 9.5 wt %, at least 10 wt %, at least 10.5 wt %, at least 11 wt %, at least 11.5 wt %, at least 12 wt %, at least 12.5 wt %, at least 13 wt %, at least 13.5 wt %, at least 14 wt %, at least 14.5 wt %, or at least 15.0 wt % PA.
34. The method of embodiment 32, wherein the BSO comprises about 9 wt %, about 9.5 wt %, about 10 wt %, about 10.5 wt %, about 11 wt %, about 11.5 wt %, about 12 wt %, about 12.5 wt %, about 13 wt %, about 13.5 wt %, about 14 wt %, about 14.5 wt %, or about 15.0 wt % PA.
35. The method of any one of embodiments 26 to 34, wherein the BSO comprises 18-30 wt % oleic acid (OA).
36. The method of embodiment 35, wherein the BSO comprises at least 18 wt %, at least 18.5 wt %, at least 19 wt %, at least 19.5 wt %, at least 20 wt %, at least 20.5 wt %, at least 21 wt %, at least 21.5 wt %, at least 22 wt %, at least 22.5 wt %, at least 23 wt %, at least 23.5 wt %, at least 24 wt %, at least 24.5 wt %, at least 25 wt %, at least 25.5 wt %, at least 26 wt %, at least 26.5 wt %, at least 27 wt %, at least 27.5 wt %, at least 28 wt %, at least 28.5 wt %, at least 29 wt %, at least 29.5 wt %, or at least 30 wt % OA.
37. The method of embodiment 35, wherein the BSO comprises about 18 wt %, about 18.5 wt %, about 19 wt %, about 19.5 wt %, about 20 wt %, about 20.5 wt %, about 21 wt %, about 21.5 wt %, about 22 wt %, about 22.5 wt %, about 23 wt %, about 23.5 wt %, about 24 wt %, about 24.5 wt %, at least 25 wt %, about 25.5 wt %, about 26 wt %, about 26.5 wt %, about 27 wt %, about 27.5 wt %, about 28 wt %, about 28.5 wt %, about 29 wt %, about 29.5 wt %, or about 30 wt % OA.
38. The method of any one of embodiment 26 to 37, wherein the BSO comprises 52-68 wt % linoleic acid (LA).
39. The method of embodiment 38, wherein the BSO comprises at least 52 wt %, at least 52.5 wt %, at least 53 wt %, at least 53.5 wt %, at least 54 wt %, at least 54.5 wt %, at least 55 wt %, at least 55.5 wt %, at least 56 wt %, at least 56.5 wt %, at least 57 wt %, at least 57.5 wt %, at least 58 wt %, at least 58.5 wt %, at least 59 wt %, at least 59.5 wt % LA, at least 60 wt %, at least 60.5 wt %, at least 61 wt %, at least 61.5 wt %, at least 62 wt %, at least 62.5 wt %, at least 63 wt %, at least 63.5 wt %, at least 64 wt %, at least 64.5 wt %, at least 65 wt %, at least 65.5 wt %, at least 66 wt %, at least 66.5 wt %, at least 67 wt %, at least 67.5 wt %, or at least 68 wt % LA.
40. The method of embodiment 38, wherein BSO comprises about 52 wt %, about 52.5 wt %, about 53 wt %, about 53.5 wt %, about 54 wt %, about 54.5 wt about 55 wt %, about 55.5 wt %, about 56 wt %, about 56.5 wt %, about 57 wt %, about 57.5 wt %, about 58 wt %, about 58.5 wt %, about 59 wt %, about 59.5 wt % LA, about 60 wt %, about 60.5 wt %, about 61 wt %, about 61.5 wt %, about 62 wt %, about 62.5 wt %, about 63 wt %, about 63.5 wt %, about 64 wt %, about 64.5 wt %, about 65 wt %, about 65.5 wt %, about 66 wt %, about 66.5 wt %, about 67 wt %, about 67.5 wt %, or about 68 wt % LA.
41. The method of embodiment 26, wherein the BSO comprises at least 1.7 wt % TQ, 9-15 wt % palmitic acid, 18-30 wt % oleic acid, and 52-68 wt % linoleic acid.
42. The method of any one of embodiments 26-41, wherein the TQ composition comprises 0.8-6.0 wt % eicosapentaenoic acid (EPA).
43. The method of embodiment 42, wherein the TQ composition comprises at least 0.8 wt %, at least 1.0 wt %, at least 1.5 wt %, at least 2.0 wt %, at least 2.5 wt %, at least 3.0 wt %, at least 3.5 wt %, at least 4.0 wt %, at least 4.5 wt %, at least 5.0 wt %, at least 5.5 wt %, or at least 6.0 wt % EPA.
44. The method of embodiment 42, wherein the TQ composition comprises about 0.8 wt %, about 1.0 wt %, about 1.5 wt %, about 2.0 wt %, about 2.5 wt %, about 3.0 wt %, about 3.5 wt %, about 4.0 wt %, about 4.5 wt %, about 5.0 wt %, about 5.5 wt %, or about 6.0 wt % EPA.
45. The method of any one of embodiment 26-44, wherein the TQ composition comprises 0.4-3.0 wt % docosahexaenoic acid (DHA).
46. The method of embodiment 45, wherein the TQ composition comprises at least 0.4 wt %, at least 0.8 wt %, at least 1.2 wt %, at least 1.6 wt %, at least 2 wt %, at least 2.4 wt %, at least 2.8 wt %, or at least 3 wt % DHA.
47. The method of embodiment 45, wherein the TQ composition comprises about 0.4 wt %, about 0.8 wt %, about 1.2 wt %, about 1.6 wt %, about 2 wt %, about 2.4 wt %, about 2.8 wt %, or about 3 wt % DHA.
48. The method of any one of embodiments 26-47, wherein the TQ composition is orally administered in a liquid formulation comprising the BSO.
49. The method of any one of embodiments 26-47, wherein the TQ composition is orally administered in a solid dosage formulation comprising the BSO.
50. The method of embodiment 49, wherein the solid dosage formulation is a tablet comprising BSO.
51. The method of embodiment 49, wherein the solid dosage formulation is a capsule comprising BSO.
52. The method of embodiment 49, wherein the solid dosage formulation is a soft capsule comprising BSO.
53. The method of embodiment 49, wherein the solid dosage formulation is a soft gel capsule comprising BSO.
54. The method of embodiment 50, wherein the tablet is an enteric tablet.
55. The method of any one of embodiments 51-53, wherein the capsule is an enteric capsule.
56. The method of embodiment 55, wherein the enteric capsule includes an enteric component incorporated directly into the capsule.
57. The method of embodiment 55 or 56, wherein the enteric capsule remains intact in gastric acid having a pH of about 3 or lower.
58. The method of embodiment 55 or 56, wherein the enteric capsule dissolves or disintegrates at a pH greater than about 5.5.
59. The method of embodiment 55 or 56, wherein the enteric capsule dissolves or disintegrates at a pH of about 6 to about 7, while remaining intact at a pH under about 5.5.
60. The method of embodiment 55 or 56, wherein the enteric capsule dissolves or disintegrates at a pH of above 7, while remaining intact at a pH under about 6.
61. The method of embodiment 49, wherein the BSO comprises at least 1.7 wt % TQ, 9-15 wt % palmitic acid, 18-30 wt % oleic acid, and 52-68 wt % linoleic acid, wherein the solid dosage formulation is an enteric capsule comprising BSO, and wherein the enteric capsule includes an enteric component incorporated directly into the capsule.
62. The method of embodiment 61, wherein the capsule is a soft gel capsule.
63. The method of embodiment 61, wherein the solid dosage formulation is NP-101 (TQ Formula).
64. The method of any one of embodiments 48-63, wherein the TQ composition is administered in a total daily amount of at least 1.5 gm, at least 2 gm, at least 2.5 gm, or at least 3 gm BSO.
65. The method of any one of embodiments 48-63, wherein the BSO in the TQ composition is administered in a total daily amount of about 1.2 gm BSO, about 1.8 gm BSO, about 2.4 gm BSO, or about 3 gm BSO.
66. The method of any one of embodiments 61-63, wherein the BSO in the TQ composition is administered in a total daily amount of about 3 gm BSO.
67. The method of any one of embodiments 48-63, wherein the TQ in the TQ composition is administered in a total daily amount of at least 25 mg, at least 35 mg, at least 40 mg, at least 45 mg, or at least 50 mg TQ; or is administered in a total daily amount of about 25 mg, about 35 mg, about 40 mg, about 45 mg, or about 50 mg TQ.
68. The method of any one of embodiments 61-63, wherein the TQ in the TQ composition is administered in a total daily amount of at least 50 mg TQ.
69. The method of embodiment 67, wherein the TQ composition is NP-101 (TQ Formula).
70. The method of embodiment 68, wherein the TQ composition is NP-101 (TQ Formula).
71. The method of embodiment 69 or 70, wherein the TQ Formula is an enteric capsule comprising BSO and an enteric component incorporated directly into the capsule.
72. The method of embodiment 71, wherein the TQ Formula is administered in 5 capsules daily, each capsule comprising 600 mg BSO.
73. The method of any one of embodiments 1-72, wherein the NEN is a pulmonary NEN.
74. The method of any one of embodiments 1-72, wherein the NEN is an extrapulmonary NEN.
75. The method of any one of embodiments 1-72, wherein the NEN is a gastroenteropancreatic NEN (GEP-NEN).
76. The method of any one of embodiments 1-72, wherein the NEN is a well-differentiated neuroendocrine tumor (NET).
77. The method of embodiment 76, wherein the NET is a high-grade (G3) NET.
78. The method of any one of embodiments 1-72, wherein the NEN is a poorly differentiated neuroendocrine carcinoma (NEC).
79. The method of embodiment 78, wherein the NEC is an extrapulmonary NEC (EP-NEC).
80. The method of embodiment 79, wherein the EP-NEC is a gastroenteropancreatic NEC (GEP-NEC).
81. The method of embodiment 79, wherein the EP-NEC is of unknown primary origin, arises primarily in the head or neck, or arises in the genitourinary tract.
82. The method of any one of embodiments 79-81, wherein the EP-NEC is a small cell type NEC (SC-NEC).
83. The method of any one of embodiments 79-81, wherein the EP-NEC is a large cell type NEC (LC-NEC).
84. The method of embodiment 78, wherein the NEC is a pulmonary NEC.
85. The method of embodiment 84, wherein the pulmonary NEC is a large cell lung cancer (LCLC).
86. The method of embodiment 84, wherein the pulmonary NEC is a small cell lung cancer (SCLC).
87. The method of any one of embodiments 1-72, wherein the NEN is a mixed neuroendocrine-non-neuroendocrine neoplasm (MiNEN).
88. The method of embodiment 87, wherein the MiNEN is a mixed neuroendocrine-nonneuroendocrine carcinoma.
89. The method of any one of embodiments 73-88, wherein the NEN is a stage I or stage II NEN. 90. The method of any one of embodiments 73-88, wherein the NEN is a stage III NEN.
91. The method of any one of embodiments 73-88, wherein the NEN is a stage IV NEN.
92. The method of any one of embodiments 80-83, wherein the EP-NEC is an advanced EP-NEC.
93. The method of embodiment 92, wherein the EP-NEC is a metastatic EP-NEC.
94. The method of embodiment 84, wherein the pulmonary NEC is an advanced pulmonary NEC.
95. The method of embodiment 84 or 94, wherein the pulmonary NEC is a metastatic pulmonary NEC.
96. The method of embodiment 88, wherein the mixed neuroendocrine-nonneuroendocrine carcinoma is an advanced MiNEN.
97. The method of embodiment 88 or 96, wherein the mixed neuroendocrine-nonneuroendocrine carcinoma is a metastatic MiNEN.
98. The method of embodiment 79 or 80, wherein the NEC is a small cell type NEC (SC-NEC).
99. The method of embodiment 79 or 80, wherein the NEC is a large cell type NEC (LC-NEC).
100. A method for treating a neuroendocrine carcinoma (NEC), comprising administering to a patient in need thereof a therapeutically effective amount of a thymoquinone (TQ) composition in combination with an immunotherapy comprising a PD-1 inhibitor and a CTLA-4 inhibitor.
101. The method of embodiment 100, wherein the PD-1 inhibitor is an anti-PD-1 antibody and the CTLA-4 inhibitor is an anti-CLTA-4 antibody.
102. The method of embodiment 101, wherein the anti-PD-1 antibody is nivolumab and the anti-CTLA-4 antibody is ipilimumab.
103. The method of any one of embodiments 100 to 102, wherein the patient has previously progressed on at least one line of cytotoxic chemotherapy.
104. The method of embodiment 103, wherein the cytotoxic chemotherapy is a platinum-based combination therapy comprising cisplatin (or carboplatin) and etoposide.
105. The method of any one of embodiments 100-104, wherein the TQ composition is orally administered in a dosage form comprising BSO.
106. The method of embodiment 105, wherein the dosage form is an enteric capsule that includes an enteric component incorporated directly into the capsule.
107. The method of embodiment 105 or 106, wherein the dosage form is NP-101 (TQ Formula).
108. The method of embodiment 107, wherein the BSO in the TQ formula comprises at least 1.7 wt % TQ.
109. The method of embodiment 107 or 108, wherein the BSO in the TQ formula comprises 9-15 wt % palmitic acid.
110. The method of any one of embodiments 107-109, wherein the BSO in the TQ formula comprises 18-30 wt % oleic acid.
111. The method of any one of embodiments 107-110, wherein the BSO in the TQ formula comprises 52-68 wt % linoleic acid.
112. The method of embodiment 107 or 108, wherein the BSO in the TQ formula comprises at least 1.7% wt % TQ, 9-15 wt % palmitic acid, 18-30 wt % oleic acid, and 52-68 wt % linoleic acid.
113. The method of embodiment 112, where the BSO further comprises 0.8-6.0 wt % eicosapentaenoic acid (EPA).
114. The method of embodiment 112 or 113, where the BSO further comprises 0.4-3.0 wt % docosahexaenoic acid (DHA).
4860-6482-8413, v. 1-63-115. The method any one of embodiments 100-114, wherein the NEC is an advanced EP-NEC.
116. The method of embodiment 100-114, wherein the NEC is a metastatic EP-NEC.
117. The method of any one of embodiments 100-114, wherein the NEC is an advanced and metastatic NEC.
118. The method of any one of embodiments 100-117, wherein the TQ composition is administered in a total daily amount comprising at least 1.5 gm, at least 2.0 gm, at least 2.5 gm, or at least 3 gm BSO.
119. The method of any one of embodiments 100-117, wherein the TQ composition is administered in a total daily amount comprising 3 gm BSO.
120. The method of any one of embodiments 100-117, wherein the TQ composition is administered in a total daily amount of at least 25 mg, at least 35 mg, at least 40 mg, at least 45 mg, or at least 50 mg TQ.
121. The method of any one of embodiments 100-117, wherein the TQ composition is administered in a total daily amount of at least 50 mg TQ.
122. The method of embodiment 119, wherein the TQ composition is NP-101 (TQ Formula).
123. The method of embodiment 121, wherein the TQ composition is NP-101 (TQ Formula).
124. The method of embodiment 122 or claim 123, wherein the TQ Formula is a soft gel enteric capsule comprising BSO and an enteric component incorporated directly into the capsule.
125. The method of embodiment 124, wherein the TQ Formula is administered in 5 capsules daily, each capsule comprising 600 mg BSO.
126. A method for treating an advanced or metastatic extra-pulmonary neuroendocrine carcinoma (EP-NEC), comprising administering to a patient in need thereof a therapeutically effective amount of a thymoquinone (TQ) composition in combination with dual immune checkpoint inhibitors (ICPIs) comprising an anti-PD-1 antibody and an anti-CTLA-4 antibody.
127. The method of embodiment 126, wherein the anti-PD-1 antibody is nivolumab and the anti-CTLA-4 antibody is ipilimumab.
128. The method of embodiment 126 or 127, wherein the patient has previously progressed on at least one line of cytotoxic chemotherapy.
129. The method of embodiment 128, wherein the cytotoxic chemotherapy is a platinum-based combination therapy comprising cisplatin (or carboplatin) and etoposide.
130. The method of any one of embodiments 126-129, wherein the TQ is orally administered in a dosage form comprising BSO.
131. The method of embodiment 130, wherein the dosage form is an enteric capsule that includes an enteric component incorporated directly into the capsule.
132. The method of embodiment 131, wherein the BSO in the TQ composition comprises at least 1.7 wt % TQ.
133. The method of embodiment 131 or 132, wherein the BSO in the TQ composition comprises 9-15 wt % palmitic acid.
134. The method of any one of embodiments 131-133, wherein the BSO in the TQ composition comprises 18-30 wt % oleic acid.
135. The method of any one of embodiments 131-134, wherein the BSO in the TQ composition comprises 52-68 wt % linoleic acid.
136. The method of embodiment 135, wherein the BSO in the TQ composition comprises 1.7% wt % TQ, 9-15 wt % palmitic acid, 18-30 wt % oleic acid, and 52-68 wt % linoleic acid.
137. The method of any one of embodiments 126-136, wherein the TQ composition is administered in a total daily amount comprising at least 1.5 gm, at least 2.0 gm, at least 2.5 gm, or at least 3 gm BSO.
138. The method of embodiment 136, wherein the TQ composition is administered in a total daily amount comprising 3 gm BSO.
139. The method of any one of embodiments 126-136, wherein the TQ composition is administered in a total daily amount of at least 25 mg, at least 35 mg, at least 40 mg, at least 45 mg, or at least 50 mg TQ.
140. The method of embodiment 139, wherein the TQ composition is administered in a total daily amount of at least 50 mg TQ.
141. The method of any one of embodiments 132-136, wherein the dosage form is NP-101 (TQ Formula).
142. The method of embodiment 141, wherein the TQ Formula is a soft gel enteric capsule comprising BSO and an enteric component incorporated directly into the capsule.
143. The method of embodiment 142, wherein the TQ Formula is administered in 5 capsules daily, each capsule comprising 600 mg BSO.
144. The method of embodiment 136, where the BSO further comprises 0.8-6.0 wt % eicosapentaenoic acid (EPA).
145. The method of embodiment 136 or 144, where the BSO further comprises 0.4-3.0 wt % docosahexaenoic acid (DHA).
146. The method of any one of embodiments 126-145, where the EP-NEC is a gastroenteropancreatic NEC (GEP-NEC).
147. The method of any of embodiments 126-145, wherein the EP-NEC patient has a median duration of response of at least 7.5 months.
148. The method of any of embodiments 126-145, wherein the EP-NEC patient has a median duration of response of about 7.5 months.
149. The method of embodiment 146, wherein the GEP-NEC patient has a median duration of response of at least 13 months.
150. The method of embodiment 146, wherein the GEP-NEC patient has a median duration of response of about 13 months.
151. The method of any one of embodiments 126-145, wherein the EP-NEC patient has a median progression free survival (PFS) of at least 2 months.
152. The method of any one of embodiments 126-145, wherein the EP-NEC patient has a median PFS of about 2 months.
153. The method of embodiment 146, wherein the GEP-NEC patient has a median PFS of at least 5.5 months.
154. The method of embodiment 146, wherein the GEP-NEC patient has a median PFS of about 5.5 months.
155. The method of any of embodiments 126-145, wherein the EP-NEC patient has at least a 30% likelihood of a 12-month PFS.
156. The method of any of embodiments 126-145, wherein the EP-NEC patient has about a 30% likelihood of a 12-month PFS.
157. The method of embodiment 46, wherein the GEP-NEC patient has at least a 50% likelihood of a 12-month PFS.
158. The method of any of embodiment 46, wherein the GEP-NEC patient has about a 50% likelihood of a 12-month PFS.
159. The method of any of embodiments 126-145, wherein the EP-NEC patient has a median OS of at least 6 months, 7 months, 8 months, 9 months, 10 months, 11 months, 12, months, 13 months, 14 months, 15 months, 16 months, 17 months, 18 months, 19 months, or 20 months.
160. The method of any of embodiments 126-145, wherein the EP-NEC patient has an OS of at least 4 months, 5 months, 6 months, 7 months, 8 months, 9 months, 10 months, 11 months, 12, months, 13 months, 14 months, 15 months, 16 months, 17 months, 18 months, 19 months, or 20 months.
161. The method of embodiments 146, wherein the GEP-NEC patient has a median overall survival (OS) of at least 7 months, 8 months, 9 months, 10 months, 11 months, 12, months, 13 months, 14 months, 15 months, 16 months, 17 months, 18 months, 19 months, or 20 months.
162. The method of any of embodiments 126-145, wherein the GEP-NEC patient has an OS of least 4 months, 5 months, 6 months, 7 months, 8 months, 9 months, 10 months, 11 months, 12, months, 13 months, 14 months, 15 months, 16 months, 17 months, 18 months, 19 months, or 20 months.
163. The method of any of embodiments 126-145, wherein the EP-NEC patient responds to treatment and has an overall survival (OS) of at least 12 months, 13 months, 14 months, 15 months, 16 months, 17 months, 18 months, 19 months, or 20 months.
164. The method of embodiment 146, wherein the GEP-NEC patient responds to treatment and has an OS of at least 12 months, 13 months, 14 months, 15 months, 16 months, 17 months, 18 months, 19 months, or 20 months.
165. The method of any one of embodiments, 1-164 wherein the patient shows an increase in CD4 positive lymphocytes in the blood during treatment.
166. The method of claim 165, wherein the increase in CD4 positive lymphocytes is an increase in helper T cells.
167. The method of any one of embodiments 1-164, wherein the patient shows an increase in CD8 positive lymphocytes in the blood during treatment.
168. The method of claim 167, wherein the increase in CD8 positive lymphocytes is an increase in cytotoxic T cells.
169. The method of any one of the preceding embodiments, wherein the patient shows an increase in CD4 and CD8 positive lymphocytes in the blood during treatment.
170. The method of claim 169, wherein the increase in CD4 positive lymphocytes is an increase in CD4 positive helper T cells and the increase in CD8 positive lymphocytes is an increase in cytotoxic T cells.

1. A method for treating an advanced or metastatic extra-pulmonary neuroendocrine carcinoma (EP-NEC), comprising administering to a patient in need thereof a therapeutically effective amount of a thymoquinone (TQ) composition in combination with dual immune checkpoint inhibitors (ICPIs) comprising an anti-PD-1 antibody and an anti-CTLA-4 antibody;
wherein the patient has previously progressed on at least one line of cytotoxic chemotherapy; and
wherein the TQ composition is orally administered in a dosage form comprising BSO.

2. The method of claim 1, wherein the BSO in the dosage form comprises at least 1.7% wt % TQ, 9-15 wt % palmitic acid, 18-30 wt % oleic acid, and 52-68 wt % linoleic acid.

3. The method of claim 2, wherein the TQ composition is administered in a total daily amount comprising at least 3 gm BSO.

4. The method of claim 3, wherein the anti-PD-1 antibody is nivolumab and the anti-CTLA-4 antibody is ipilimumab.

5. The method of claim 3, wherein the cytotoxic chemotherapy comprises a platinum-based combination therapy.

6. The method of claim 3, wherein the dosage form is an enteric capsule that includes an enteric component incorporated directly into the capsule.

7. The method of claim 3, where the BSO further comprises 0.8-6.0 wt % eicosapentaenoic acid (EPA).

8. The method of claim 3, where the BSO further comprises 0.4-3.0 wt % docosahexaenoic acid (DHA).

9. The method of claim 3, where the BSO further comprises 0.8-6.0 wt % eicosapentaenoic acid (EPA) and 0.4-3.0 wt % docosahexaenoic acid (DHA).

10. The method of claim 3, wherein the EP-NEC patient has a median duration of response of at least 7.5 months.

11. The method of claim 3, wherein the EP-NEC patient has a median OS of at least 6 months.

12. The method of claim 3, wherein the EP-NEC patient has at least a 30% likelihood of a 12-month PFS.

13. The method of claim 3, wherein the EP-NEC patient responds to treatment and survives for at least 12 months.

14. The method of claim 3 where the EP-NEC is a gastroenteropancreatic NEC (GEP-NEC).

15. The method of claim 14, wherein the GEP-NEC patient has a median duration of response of at least 13 months.

16. The method of claim 14, wherein the GEP-NEC patient has a median OS of at least 7 months.

17. The method of claim 14, wherein the GEP-NEC patient has a median PFS of at least 5.5 months.

18. The method of claim 14 wherein the GEP-NEC patient has at least a 50% likelihood of a 12-month PFS.

19. The method of claim 14, wherein the GEP-NEC patient responds to treatment and survives for at least 12 months.

20. The method of claim 3, wherein the patient responds to treatment and shows an increase in blood of CD4 positive helper T cells and CD8 positive cytotoxic T cells during treatment.
